(19) United States
(12) Patent Application Publication
(10) Pub. No.: US 2025/0049363 A1
(43) Pub. Date: Feb. 13, 2025

(54) METHOD AND SYSTEM FOR OBTAINING MEASUREMENT OF COGNITIVE PERFORMANCE

(71) Applicant: Altoida, Inc., Washington, DC (US)

(72) Inventors: Maximillian Bulger, Schechen (DE); Robbert Harms, Nijmegen (NL); Ioannis Tarnanas, Washington, DC (US)

(21) Appl. No.: 18/570,900

(22) PCT Filed: Jun. 8, 2022
(86) PCT No.: PCT/IB2022/055333

§ 371 Date: Dec. 15, 2023

Related U.S. Application Data
(60) Provisional application No. 63/211,953, filed on Jun. 17, 2021.

(51) Int. Cl. A61B 5/16; A61B 5/00; A61B 5/11

(52) U.S. Cl. CPC A61B 5/162; A61B 5/1124; A61B 5/4088; A61B 5/7475; A61B 2562/0219

(57) ABSTRACT

A method and system for obtaining a measurement of cognitive performance in an individual, provide for obtaining a measure of at least one of the following activity parameters for the individual: 1) spatial memory accuracy; 2) planning accuracy; 3) ability to carry out dual-task interactions while navigating to a goal, wherein omission of the dual-task interactions is measured; 4) perseverations of incorrect dual-task interactions while navigating to a goal; 5) total time required by the individual to complete a navigation route; 6) upper extremity neuro-motor parameters; 7) reaction time of dual-task interactions; and 8) reaction time of the idle state of the individual; receiving the measures obtained into an algorithm; and computing a functional impairment score for the individual on the basis of baseline measurements obtained from a population of healthy individuals. The measurements are obtained using an app on an electronic portable device. The system and method may include an application executable by a user device to generate a gamified environment, to receive a first user input or set of user inputs at a first time and to transform said first user input or set of user inputs into a first measurement or set of measurements, and to receive a second user input or set of user inputs at a second time and to transform said second user input or set of user inputs into a second measurement or set of measurements; configured to receive said first measurement or set of measurements and compute a first functional impairment score and to receive said second measurement or set of measurements and compute a second functional impairment score; and configured to compare said second functional impairment score with said first functional impairment score and determine a magnitude and/or a speed of change in said functional impairment scores.

### TECHNICAL FIELD

This invention relates to a method of obtaining a measurement of cognitive performance in an individual and a computer implemented system for obtaining a measurement of cognitive performance in an individual.

### BACKGROUND TO THE INVENTION

More than 50 million people in the world today are affected by dementia, most suffering from Alzheimer's disease (AD). In both developed and developing nations, AD has tremendous impact on the affected individuals, caregivers, and society.

Dementia can be defined as a clinical syndrome characterised by a cluster of symptoms and signs manifested by difficulties in memory, disturbances in language and other cognitive functions, changes in behaviours and impairments in activities of daily living. AD is the most common cause of dementia, accounting for up to 75% of all dementia cases, and is a progressive neurodegenerative disorder.

AD is a degenerative brain disease caused by brain changes that lead to dementia symptoms that gradually worsen over time. Early symptoms include difficulties remembering information. As AD progresses, symptoms get more severe and include disorientation, confusion and behaviour changes. Eventually, speaking, swallowing and walking become difficult. While there exist prescription drugs to treat AD symptoms, there is currently no way to prevent, cure or even slow AD, which is ultimately fatal.

AD is characterised by a preclinical phase, lasting years, during which progressive neurodegeneration in the brain occurs before typical clinical symptoms (e.g. cognitive deficits and subtle cognitive disturbances) become detectable (Bäckman et al. (2001)). Theoretically, detection of AD at an early stage may provide an opportunity for implementing therapeutic intervention to delay more effectively its progression to clinical dementia.

However, there remains a challenge as to how to identify individuals during the preclinical phase of the disease, although some clinical markers, neuroimaging biomarkers, and biochemical markers have been investigated (DeKosky & Marek (2003)).

First, numerous studies have suggested that deficits in specific cognitive domains such as episodic memory and verbal ability are conceivable up to 10 years before the dementia syndrome can be clinically diagnosed, with a more evident decline occurring over the final few years. The term “mild cognitive impairment” has been used in clinical settings to identify individuals with isolated memory loss (i.e. “amnestic” type MCI), which is more likely to represent the preclinical phase of AD. However, population-based follow up studies have frequently shown that individuals with MCI represent a very heterogeneous group in terms of prognosis (Palmer et al. (2002)); although elderly persons with MCI had increased risk of progressing to dementia, a substantial proportion remained stable or even reverted to normal during the next few years. Where the mild cognitive impairment is in fact due to AD, this is known as Prodromal AD.

Second, biochemical markers in serum and cerebrospinal fluid such as β-amyloid and t-protein have been proposed for early detection of AD, but these markers are not sufficiently reliable in making diagnosis of AD in the preclinical phase (Blennow et al. (2006); DeKosky & Marek (2003)).

Finally, during the last decade neuroimaging has emerged as a useful tool to define AD at both preclinical and early clinical phases of the disease. For example, the amyloid positron emission tomography imaging tracer ligands offer the opportunity to measure β-amyloid in the brain in vivo, which provides the possibility of early diagnosis and of monitoring the course of anti-amyloid therapy in AD (Nordberg (2007); Forsberg et al. (2008)). Furthermore, the medial-temporal lobe atrophy seen on volumetric MRI has been used in the identification of MCI and early AD as well as in the assessment of progression of MCI and early AD (Dubois et al. (2007); Ridha et al. (2007)). However, these tests are currently limited to research applications due to their cost and invasive nature. These limitations preclude repeated and frequent use to test an individual and specifically in the early pre-symptomatic stage (Kourtis et al. (2019)).

Mobile and wearable digital consumer technology have the potential to overcome the above limitations, and their application in AD detection has become an area of increased interest. For example, the applicant's earlier patent applications (published as WO 2010/075481, WO 2016/157093, and WO 2020/049470, the contents of which are incorporated herein by reference) disclose the use of mobile devices to enable tests for determining a cognitive state of a user.

Longitudinal measures of cognitive performance are important for evaluating preclinical markers and prodromal periods of cognitive impairment and dementia, as well as for monitoring disease progression. Current techniques for assessing cognitive decline are often based on cross-sectional assessments (i.e., observations at a specific point in time). However, cross-sectional assessments are of limited value in capturing an individuals' global cognitive function and may not accurately predict future cognitive performance and risk of cognitive decline due to high intra-individual variability in cognitive performance (Mungas et al. (2010)).

Conventional cross-sectional neuropsychological assessments of cognition are vulnerable to several confounders that can affect an individual's assessment performance such as motivation, attention, mood, and testing environment. In turn, the unreliable nature of such neuropsychological assessments has negative consequences for clinical care as it is used for prognosis, diagnosis, and eventually, treatment of brain-related diseases such as the dementia family of diseases (e.g. AD). Conventional neuropsychological assessments for AD are lengthy, unreliable, and inaccurate at capturing MCI, and present significant variability across different contexts and times, especially after repeated measurements. Reasons for this variability are multiple, such as participants' motivation, attention, mood, anxiety levels, sleep quality the night before the assessment, and testing environment. Such variability can lead to inaccurate diagnosis and inappropriate treatment, for example, by giving the false impression that a patient's cognition has improved at a follow up visit. Limitations in conventional cross-sectional neuropsychological assessments highlight a significant clinical and research gap in cognitive assessment across the full spectrum of individuals from healthy cognitive function to dementia.

### SUMMARY OF THE PRESENT INVENTION

The present invention seeks to provide an improved method of obtaining a measurement of cognitive performance in an individual and an improved computer implemented system for obtaining a measurement of cognitive performance in an individual.

According to an aspect of the present invention, there is provided a method of obtaining a measurement of cognitive performance in an individual, the method including obtaining a measure of at least one of the following activity parameters for the individual:
1) spatial memory accuracy;
2) planning accuracy;
3) ability to carry out dual-task interactions while navigating to a goal, wherein omission of the dual-task interactions is measured;
4) perseverations of incorrect dual-task interactions while navigating to a goal;
5) total time required by the individual to complete a navigation route;
6) upper extremity neuro-motor parameters;
7) reaction time of dual-task interactions; and
8) reaction time of the idle state of the individual;
   receiving the measures obtained into an algorithm; and
computing a functional impairment score for the individual on the basis of baseline measurements obtained from a population of healthy individuals.

The method is preferably computer-implemented. It may be carried out using a computer implemented system or a computer system as set out below.

The information required for obtaining the measurements is preferably received via an input interface of a mobile device. The measurements are preferably received by a processor and configured to execute the algorithm to compute, using said measurements, a functional impairment score indicative of cognitive performance in the individual. The functional impairment score is preferably accessible remotely by a third party and displayable at an information output device.

The measurements may be obtained using an app on an electronic portable device. The portable electronic device may be a smart phone or tablet for example.

According to another aspect of the present invention, there is provided a computer implemented system for obtaining a measurement of cognitive performance in an individual, said system including:
an input interface configured to receive measurements from a remote source on an individual in respect of at least one of the following activity parameters:
1) spatial memory accuracy;
2) planning accuracy;
3) ability to carry out dual-task interactions while navigating to a goal, wherein omission of the dual-task interactions is measured;
4) perseverations of incorrect dual-task interactions while navigating to a goal;
5) total time required by the individual to complete a navigation route;
6) upper extremity neuro-motor parameters;
7) reaction time of dual-task interactions; and
8) reaction time of the idle state of the individual;
a processor configured to receive said measurements and configured to execute computer program code to compute, using said measurements, a functional impairment score indicative of cognitive performance in the individual.

The system may include:
an input interface configured to receive a first measurement or set of measurements obtained at a first time and a second measurement or set of measurements obtained at a second different time in respect of the activity parameter or parameters;
a processor configured to receive said first measurement or set of measurements and configured to execute computer program code to compute, using said first measurement or set of measurements, a first functional impairment score indicative of cognitive performance in the individual, and configured to receive said second measurement or set of measurements and configured to execute computer program code to compute, using said second measurement or set of measurements, a second functional impairment score;
the processor configured to compare said second functional impairment score with said first functional impairment score and determine a magnitude and/or a speed of change in said functional impairment scores.

The processor may be configured to determine both a magnitude and a speed of change in the functional impairment scores for that individual to calculate a composite (or overall) score.

According to another aspect of the present invention, there is provided a computer system for obtaining a measurement of cognitive performance in an individual, said system including:
an application executable by a user device to generate a gamified environment, to receive user inputs and to transform said inputs into measurements for the individual of at least one of the following activity parameters:
1) spatial memory accuracy;
2) planning accuracy;
3) ability to carry out dual-task interactions while navigating to a goal, wherein omission of the dual-task interactions is measured;
4) perseverations of incorrect dual-task interactions while navigating to a goal;
5) total time required by the individual to complete a navigation route;
6) upper extremity neuro-motor parameters;
7) reaction time of dual-task interactions; and
8) reaction time of the idle state of the individual;
a system configured to receive said measurements and compute a functional impairment score indicative of cognitive performance in the individual.

The system may include:
an application executable by a user device to generate a gamified environment, to receive a first user input or set of user inputs at a first time and to transform said first user input or set of user inputs into a first measurement or set of measurements, and to receive a second user input or set of user inputs at a second time and to transform said second user input or set of user inputs into a second measurement or set of measurements;
a system configured to receive said first measurement or set of measurements and compute a first functional impairment score and to receive said second measurement or set of measurements and compute a second functional impairment score;
the system configured to compare said second functional impairment score with said first functional impairment score and determine a magnitude and/or a speed of change in said functional impairment scores.

The system may be configured to determine both a magnitude and a speed of change in the functional impairment scores for that individual to calculate a composite (or overall) score.

A system may be configured to receive said measurements and compute the functional impairment score and/or the composite score remotely from the electronic portable device or user device. For example, the electronic portable device or other user device may upload the measurements via the internet to a remote system, where the processing/computation are carried out.

Preferably, a plurality of activity parameters is measured.

In an embodiment, at least the upper extremity neuro-motor parameters are measured. For example, motion agility, speed of motion, and/or smoothness of motion may be measured.

Preferably at least three activity parameters are measured or at least four activity parameters are measured or at least five activity parameters are measured.

Advantageously, including measurement of additional activity parameters improves the accuracy of the functional impairment score.

In an embodiment, particularly useful where a prediction of conversion from MCI to AD is the goal, at least the following activity parameters are measured:
spatial memory accuracy;
ability to carry out dual-task interactions while navigating to a goal, wherein omission of the dual-task interactions is measured;
perseverations of incorrect dual-task interactions while navigating to a goal;
upper extremity neuro-motor parameters; and
reaction time of the idle state of the individual.

In a most preferred embodiment, all eight of the activity parameters are measured.

The algorithm is executed to calculate metrics belonging to the activity parameters.

In an embodiment, a plurality of metrics belonging to the activity parameters is calculated; the metrics are mapped to a plurality of cognitive domains; and a percentile rank score for each cognitive domain is calculated.

The metrics are generally calculated on the basis of an algorithm, which may be or include one or more of signal analysis, sensor-fusion, algebraic integration, Fourier analysis or wavelet analysis.

The cognitive domains to which the metrics are mapped may include at least one of perceptual motor coordination, complex attention, cognitive processing speed, inhibition, flexibility, visual perception, planning, prospective memory, and spatial memory.

Hand movements of the individual may be assessed to obtain at least one of the measurements. This may include testing speed and/or accuracy of the individual's hand movements.

The individual's hand movements may be assessed by displaying an image to the individual and assessing the individual's ability to trace or tap on the image. The image may be displayed on the screen of a portable electronic device or other user device.

The individual's ability to navigate may be assessed to obtain at least one of the measurements. This could be achieved by assessing the individual's ability to navigate includes the individual placing and retrieving a plurality of objects.

The individual's ability to execute tasks may be assessed to obtain at least one of the measurements.

Assessing the individual's ability to execute tasks may include assessing their ability to carry out subtasks in an exact order.

Assessing the individual's ability to navigate or execute tasks may include distracting the individual during the assessment.

In an embodiment, spatial memory accuracy is determined by measuring the number of items correctly selected by the individual in a navigation assessment or by analysing the complexity of the path taken by the individual in a navigation assessment. For example, path complexity could be measured on the basis of the number of turns made by a subject whilst performing the test: fewer turns indicates a more direct line to their goal corresponding to a higher spatial memory.

Planning accuracy may be determined by measuring the correct prospective memory task execution by an individual in a task execution assessment.

The upper extremity neuro-motor parameters include motion agility, speed of motion, and/or smoothness of motion. These may be derived from signal processing of 3D acceleration data provided on a portable electronic device.

Reaction time of dual-task interactions may be measured as the time elapsed between stimulus being provided to the individual and response from the individual.

The reaction time of idle state is measured as the time elapsed between a patient idle state and the next immediate interaction response in the dual-task exercise. This could be considered a measure of reaction time, for example the time taken to react to a distraction signal (such as a high pitched tone) during a task.

The method may be carried out a plurality of times by the individual, for example at approximately monthly intervals. The method is preferably carried out at least three times, at least four times, at least five times or at least six times.

A first functional impairment score obtained from the individual may be compared with a second functional impairment score obtained from the individual at a different time, and a magnitude and/or a speed of change in said functional impairment scores for that individual may be determined.

In an embodiment, both a magnitude and a speed of change in the functional impairment scores for that individual are determined, and a composite score is calculated therefrom.

The cognitive impairment score or composite score may be computed in a system configured to receive the measurements and compute the functional impairment score or composite score remotely from the electronic portable device or user device, wherein the system includes an information output accessible by a third party remotely from the electronic portable device or user device.

The individual may have mild cognitive impairment and, based on the cognitive impairment score or composite score, a prediction of whether the individual with mild cognitive impairment will convert to Alzheimer's Disease may be made. The individual may be diagnosed with mild cognitive impairment as a result of the test, or may have been previously diagnosed with mild cognitive impairment prior to taking the test.

Use of a cognitive impairment score or composite score obtainable by a method or by a system as specified above to predict conversion of an individual that has previously been diagnosed with mild cognitive impairment to Alzheimer's Disease, to diagnose Alzheimer's Disease, or to diagnose mild cognitive impairment.

If, on the basis of the cognitive impairment score or composite score, an individual with mild cognitive impairment is predicted to convert to Alzheimer's Disease, information relating to a pharmaceutical or other intervention may be provided by the information output.

The suggested intervention may be a pharmaceutical intervention and the information may relate to the identity of a specific drug to be administered to the individual.

The drug may be a cholinesterase inhibitor (such as donepezil, rivastigmine or galantamine), memantine (optionally in combination with a cholinesterase inhibitor), a monoclonal antibody (such as Aducanumab (Aduhelm), BAN2401, gantenerumab (optionally in combination with solanezumab), solanezumab (optionally in combination with gantenerumab), a sigma-1 receptor agonist (optionally also M2 autoreceptor antagonist or NMDA receptor antagonist, such as ANAVEX2 (blarcamesine), AVP-786 or AXS-05), an SV2A modulator (such as AGB101 (low-dose levetiracetam), a mast-cell stabiliser (such as ALZT-OP1 (cromolyn+ibuprofen)), an anti-inflammatory (such as ALZT-OP1 (cromolyn+ibuprofen)), a RAGE antagonist (such as azeliragon), a glutamate modulator (such as BHV4157 (troriluzole)), a D2 receptor partial agonist (such as brexpiprazole), serotonin-dopamine modulator (such as brexpiprazole), an amyloid vaccine (such as CAD106), a bacterial protease inhibitor (such as COR388), a selective serotonin reuptake inhibitor (such as escitalopram), an antioxidant (such as Ginkgo biloba), a plant extract (such as Ginkgo biloba), an alpha-2 adrenergic agonist (such as guanfacine), an omega-3 fatty acid (such as icosapent ethyl (IPE), which is a purified form of eicosapentaenoic acid), an angiotensin II receptor blocker (such as losartan), a calcium channel blocker (such as amiodipine), a cholesterol agent (such as atorvastatin), a combination of an angiotensin II receptor blocker (such as losartan), a calcium channel blocker (such as amiodipine), a cholesterol agent (such as atorvastatin) with or without exercise, a tyrosine kinase inhibitor (such as masitinib), an insulin sensitiser (such as metformin), a dopamine reuptake inhibitor (such as methylphenidate), an alpha-1 antagonist (such as mirtazapine), an acetylcholinesterase inhibitor (such as octohydro-aminoacridine succinate), a ketone body stimulant (such as tricaprilin), a caprylic triglyceride (such as tricaprilin), a Tau protein aggregation inhibitor (such as AADvac1 or TRx0237 (LMTX)), a positive allosteric modulator of GABA-A receptors (Zolpidem and zoplicone), or BPDO-1603, or combinations of any of these to be administered either together or separately.

Where the individual shows a low score in visuospatial function, a prescription of memantine/donepezil) may be suggested. Where the individual shows a low score in executive function, a prescription of metformin may be suggested. Where the individual shows a low score for perpetual motor co-ordination, a prescription of TRx0237 may be suggested.

A prescription of Aducanumab (Aduhelm) may be suggested.

The suggested intervention may be a pharmaceutical intervention and the information may relate to the frequency and/or dose of the pharmaceutical intervention or specific drug to be administered to the individual.

The individual may have been previously diagnosed with mild cognitive impairment and the information relating to a pharmaceutical or other intervention provided by the information output relates to whether a previously prescribed intervention is effective in that individual.

According to another aspect of the present invention, there is provided a method of diagnosing Alzheimer's Disease including a method as set out above, further including the step of making a diagnosis of Alzheimer's Disease on the basis of the functional impairment score or the composite score.

According to another aspect of the present invention, there is provided a method of diagnosing mild cognitive impairment including a method as set out above, further including the step of making a diagnosis of Alzheimer's Disease on the basis of the functional impairment score or the composite score.

According to another aspect of the present inventions, there is provided a system for diagnosing Alzheimer's Disease including a system as set out above, wherein the processor or system is operable to make a diagnosis of Alzheimer's Disease on the basis of the functional impairment score or the composite score.

According to another aspect of the present invention, there is provided a system for diagnosing mild cognitive impairment including a system as set out above, wherein the processor or system is operable to make a diagnosis of Alzheimer's Disease on the basis of the functional impairment score or the composite score.

In practice the methods and systems described herein will typically be used in individuals previously diagnosed with mild cognitive impairment. The output will be used as an adjunct to other diagnostic evaluations and is intended to identify whether the MCI is due to AD or not (i.e. whether the individual has Prodromal AD). It may be used to predict whether an individual with mild cognitive impairment will go on to develop dementia, in particular dementia caused by Alzheimer's disease.

However, wider uses may be envisaged. For example, they could be used to identify MCI (e.g. MCI due to AD) in an individual that was unaware of any impairment.

### BRIEF DESCRIPTION OF THE DRAWINGS

Preferred embodiments are now described, by way of example only, with reference to and as illustrated in the accompanying drawings:

FIGS. 1 to 6 show examples of a user interface for a Motor Test;

FIGS. 7 to 16 show examples of a user interface for a Back-in-Time task;

FIGS. 17 to 23 show examples of a user interface for a Day-Out-Task;

FIG. 24 illustrates an exemplary login screen of and embodiment of dashboard for accessing test results;

FIG. 25 illustrates an exemplary Search field for subject test results;

FIG. 26 illustrates an exemplary overview of a subject's test results;

FIG. 27 shows an exemplary PDF report for Subject 2 from FIG. 26;

FIG. 28 shows receiver operating characteristic (ROC) curves of classifier;

FIG. 29 shows the variable dependencies identified in the chosen dataset;

FIG. 30 shows feature importance of MMSE, FAQ and DMs in ADNI data of the best logistic regression estimator to classify subjects into cognitively normal and MCI;

FIG. 31 shows ROC curves of classifier for another embodiment;

FIG. 32 shows SHAP performance (impact on output);

FIG. 33 provides an illustration of dispersion. Left: Individual patient data over time. Right: Patient performance dispersion (dots) at different time points (A, B, C) represented in population mean (line) picked up using the taught (top curve) and conventional neuropsychological assessments (bottom curve). SD is related to the dispersion of a given subject over time (LTRS). Dashed line: true dispersion;

FIG. 34 shows schematic illustrations of the LTRS (top) and LDVS (bottom) (numbers 17.3 and 41.5 are random examples). The left third of the line indicates low risk, the middle third indicates medium risk, and fight third indicates high risk;

FIG. 35 shows a combined longitudinal risk matrix obtained from the two measures shown in FIG. 34 (the overlapping areas allow for a more nuanced interpretation);

FIG. 36 shows an exemplary intra-individual variability score;

FIG. 37 shows dispersion index based on LTRS and neuropsychological tests plotted for the three different groups translated into Standard Deviation;

FIG. 38 shows dispersion index plotted across tasks, showing group intra-individual standard deviation (iSD) for the healthy controls (A), MCI (B) and AD (C) groups; and

FIG. 39 is a schematic diagram of an embodiment of apparatus configured to implement a system and method as taught herein.

### DESCRIPTION OF THE PREFERRED EMBODIMENTS

Early clinical recognition of AD is critical so physicians can treat the subject with prescription drugs to ease the symptoms and associated burdens of the disease, and caretakers can manage the changes in cognitive function, mood and personality. Early detection could also create opportunities for participation in clinical trials. However, there is currently a lack of tools to aid physicians in assessing cognitive function when diagnosing AD.

Currently, physicians rely on a number of mental and neuropsychological tests to assess symptoms associated with AD, such as decline in memory, abstract thinking, problem-solving, language usage, and other cognitive skills. For example, physicians employ techniques such as the Disease Assessment Scale-Cognitive Subscale (ADAS-Cog), the mini-mental state examination (MMSE), or the clock drawing test to assess the level of cognitive dysfunction in subjects with AD. Moreover, meta-analysed data from longitudinal studies are showing that a full neuropsychological assessment can strongly contribute to predicting dementia, while individuals are still in the MCI phase.

Although early clinical recognition of AD is critical, there are currently no tools to aid in the assessment of impaired cognitive function, in particular in individuals with MCI, to predict progression to AD, to assist a physician in the diagnosis of AD. Current assessment methods (such as those set out above) are onerous and often taxing for subjects.

The present application describes a computerised cognitive assessment aid, which provides a measurement of cognitive performance to aid in the assessment of impaired cognitive function to assist a physician in the prediction of and diagnosis of AD. The device is used for the purpose of identifying a potential decline in cognitive function in an adult subject relative to baseline test performance of other adults without AD, so that subjects with impaired cognitive function can be referred for further testing where warranted. The system disclosed herein is an algorithm-based software application that runs on various hardware platforms (typically a portable electronic device such as a tablet or smart phone). In preferred embodiments, the device includes functions (1) for the graphical user interface (GUI); (2) to administer a battery of motor, visual, perceptual, and memory tests; and (3) to support real-time test report generation, printing, and archiving.

The preferred embodiment is configured as an application (app) designed to be run on a portable electronic device such as a tablet or smartphone. The app is able to run a series of tests to be undertaken by a subject to evaluate, for example, perceptual motor coordination, complex attention, cognitive processing speed, inhibition, flexibility, visual perception, planning, prospective memory, and/or spatial memory. The tests may include a Motor Test to assess the hand movements of the subject. The tests may further include so-called Back-in-Time task and Day-Out-Task to assess the subject's ability to navigate and/or carry out tasks in a certain order.

The system is configured to require the user to carry out the various tests provided to them by the app on their portable electronic device. The app records the results of the tests in the form of a collection of activity parameters, including at least one of the following:
1) spatial memory accuracy;
2) planning accuracy;
3) ability to carry out dual-task interactions while navigating to a goal, wherein omission of the dual-task interactions is measured;
4) perseverations of incorrect dual-task interactions while navigating to a goal;
5) total time required by the individual to complete a navigation route;
6) upper extremity neuro-motor parameters;
7) reaction time of dual-task interactions; and
8) reaction time of the idle state of the individual.

The results are then provided to a processor, which may be remote from the individual's device. An algorithm is executed to calculate metrics belonging to the activity parameters measured. The metrics are mapped to a various cognitive domains and a percentile rank score for each cognitive domain may be calculated. From these a functional impairment score indicative of cognitive performance in the individual is computed. The score (and other related information, for example the scores relating to each individual cognitive domain) can then be accessed by a physician or other health care professional from a remote location, such as a desktop computer at their medical facility.

The system provides healthcare professionals an objective measurement of cognitive performance and can be used as an adjunctive tool to aid in evaluating perceptual and memory function in individuals.

For the individual, this system offers a personalised cognitive profile, which could be applicable also to family members. It provides time to investigate options to help mitigate the risk of cognitive decline, and early identification of subjects for clinical trial participation.

The preferred system further provides the ability to measure cognitive function to aid in the diagnostic assessment of specific diseases such as AD with a non-invasive, hand-held software device. It facilitates early intervention and management of AD with available pharmaceutical options. Due to the non-invasive nature of the system, frequent assessments are possible, allowing multiple measurements to be taken over time, which can better reflect the overall situation of the subject versus a snapshot in time.

Such longitudinal use of the system can assist in monitoring an individual's brain health over time. For example, a healthy individual can be monitored for development of MCI. An individual with MCI can be monitored for deterioration in cognitive function. The preferred embodiment of system has also been shown to be highly predictive of MCI individuals who will later convert to AD, and enables interventions to prevent or delay onset of dementia. The system can thus be used to predict conversion from MCI to AD to help a physician decide upon and prescribe a drug or other intervention. In view of the detailed information that the system is able to obtain from the user, it can also be used to suggest specific drugs for a given individual based on the results obtained.

In one embodiment, an individual can carry out the tests on a portable electronic device, for example a smartphone, at home. Their performance results in a score. By repeating the tests, for example on a daily, weekly or monthly basis, the individual's brain health can be monitored. Both the magnitude and the speed of any deterioration over time can be monitored. For an apparently healthy individual, an indication of mild cognitive impairment may be detected, and the system may suggest to the individual's physician an appropriate intervention to improve/prevent further deterioration, for example in a particular cognitive domain. Furthermore, the system can be useful in monitoring the effectiveness of a drug treatment in an individual that has been previously diagnosed with MCI. The system may quickly identify a therapeutic treatment or other intervention that is no longer effective, and an improved drug or other treatment can be suggested.

Drugs that might then be prescribed to slow or prevent further deterioration, or to treat symptoms might include a cholinesterase inhibitor (such as donepezil, rivastigmine or galantamine), memantine (optionally in combination with a cholinesterase inhibitor), a monoclonal antibody (such as Aducanumab (Aduhelm), BAN2401, gantenerumab (optionally in combination with solanezumab), solanezumab (optionally in combination with gantenerumab), a sigma-1 receptor agonist (optionally also M2 autoreceptor antagonist or NMDA receptor antagonist, such as ANAVEX2 (blarcamesine), AVP-786 or AXS-05), an SV2A modulator (such as AGB101 (low-dose levetiracetam), a mast-cell stabiliser (such as ALZT-OP1 (cromolyn+ibuprofen)), an anti-inflammatory (such as ALZT-OP1 (cromolyn+ibuprofen)), a RAGE antagonist (such as azeliragon), a glutamate modulator (such as BHV4157 (troriluzole)), a D2 receptor partial agonist (such as brexpiprazole), serotonin-dopamine modulator (such as brexpiprazole), an amyloid vaccine (such as CAD106), a bacterial protease inhibitor (such as COR388), a selective serotonin reuptake inhibitor (such as escitalopram), an antioxidant (such as Ginkgo biloba), a plant extract (such as Ginkgo biloba), an alpha-2 adrenergic agonist (such as guanfacine), an omega-3 fatty acid (such as icosapent ethyl (IPE), which is a purified form of eicosapentaenoic acid), an angiotensin II receptor blocker (such as losartan), a calcium channel blocker (such as amiodipine), a cholesterol agent (such as atorvastatin), a combination of an angiotensin II receptor blocker (such as losartan), a calcium channel blocker (such as amiodipine), a cholesterol agent (such as atorvastatin) with or without exercise, a tyrosine kinase inhibitor (such as masitinib), an insulin sensitiser (such as metformin), a dopamine reuptake inhibitor (such as methylphenidate), an alpha-1 antagonist (such as mirtazapine), an acetylcholinesterase inhibitor (such as octohydro-aminoacridine succinate), a ketone body stimulant (such as tricaprilin), a caprylic triglyceride (such as tricaprilin), a Tau protein aggregation inhibitor (such as AADvac1 or TRx0237 (LMTX)), a positive allosteric modulator of GABA-A receptors (Zolpidem and zoplicone), or BPDO-1603, or combinations of any of these to be administered either together or separately.

Depending on the results, the system may suggest a pharmaceutical intervention, change to an already implemented pharmaceutical intervention (such as change to a dosage or administration regime), and/or may indicate whether or not an intervention continues to be effective.

The system also offers the possibility to a physician to investigate scores obtained in individual areas of the tests in order to determine an optimal intervention for that individual.

Described below are possible implementations of the method and system, which may be carried out using a portable electronic device such as the user's smart phone or tablet.

### Example 1

### A. User Tasks

The user is presented with a series of visual and auditory stimuli sequentially and simultaneously and their ability to respond to variations of audio and visual stimuli is measured. The subject is asked to execute three tasks: Motor Test, Back-in-Time, and Day-Out-Task. These three types of test of varying difficulty characterise the subject's performance in each of the tested functional domains. The tests are conducted in one session with a short break (30 seconds) between tests.

a. Motor Test:

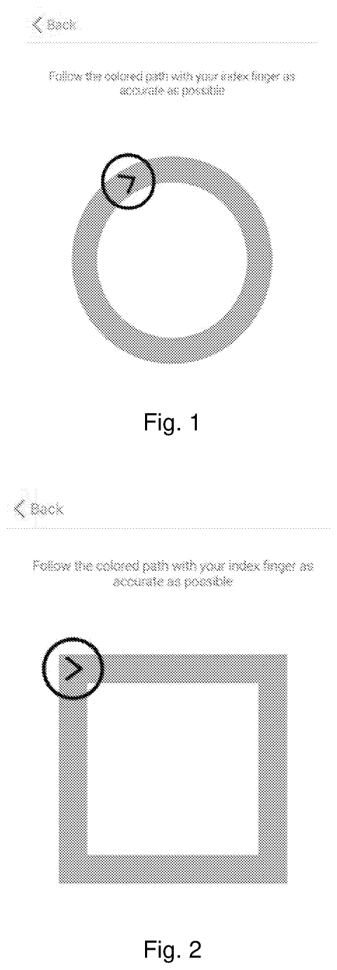

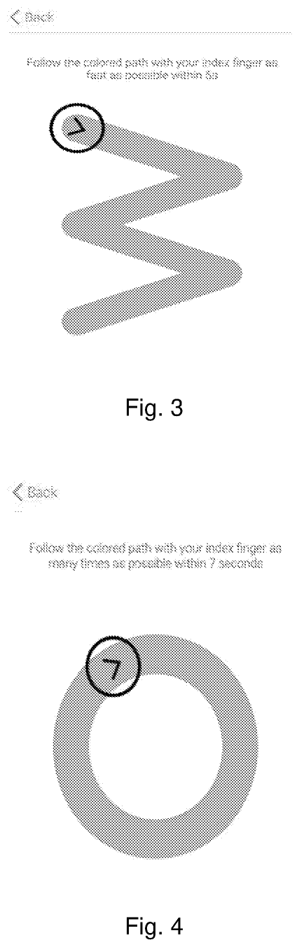

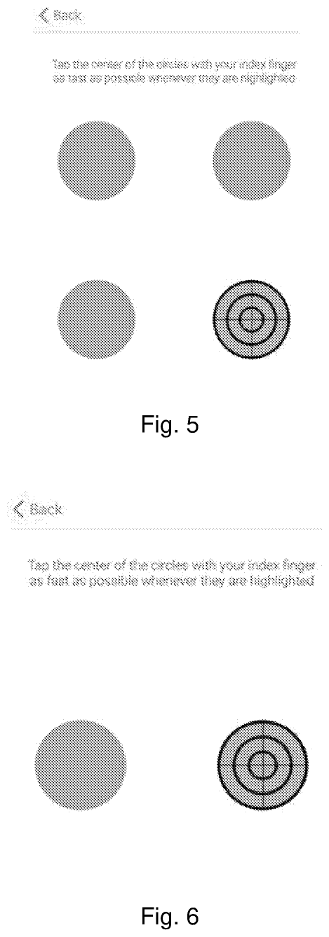

The Motor Test comprises three subsequent task types asking the subject to perform hand motion tests on the screen. FIGS. 1 to 6 show illustrative representations of the test on the screen of a portable electronic device. The first type asks the subject to follow a coloured path (FIGS. 1 and 2) as accurately and fast as possible. The second type (FIGS. 3 and 4) adds a time limit. The third type asks the subject to tap on a target object as accurately and fast as possible whenever it appears in the presence of distractor objects (green versus grey circles in FIGS. 5 and 6).

b. Back-in-Time:

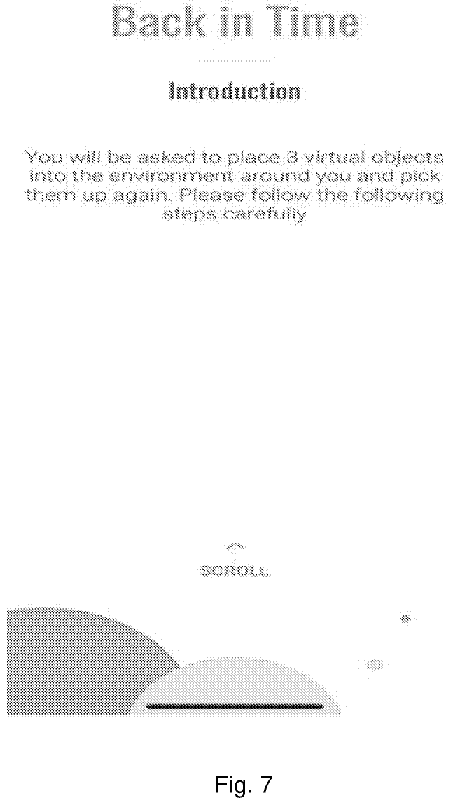

In the “Back-in-Time” test, augmented reality is used to test the subject's ability to place and retrieve a series of virtual objects, whilst being distracted by an audio signal. FIGS. 7 to 16 illustrate what the subject will see on the screen of their portable electronic device.

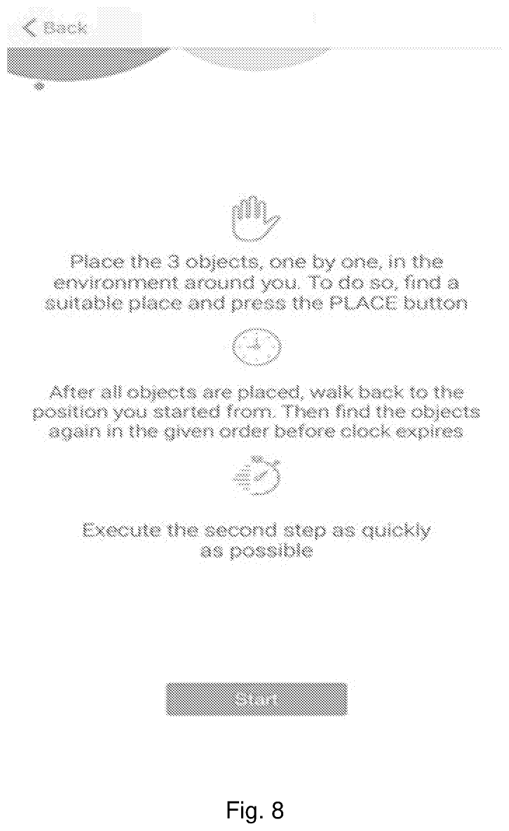

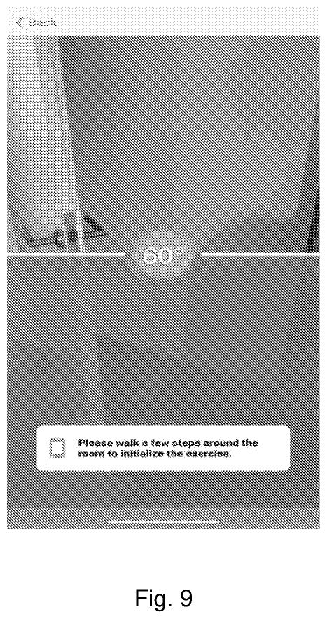

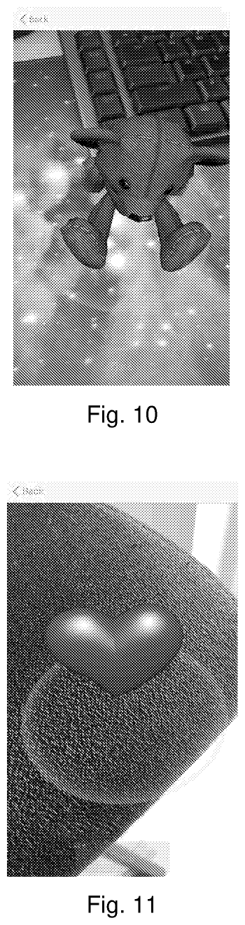

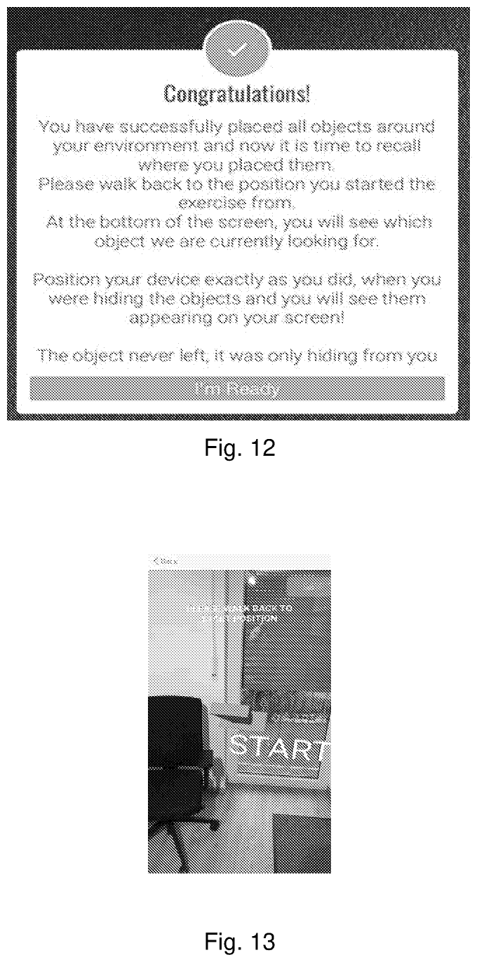

The subject is instructed to place three virtual objects into a suitable place in their real environment using augmented reality (see FIGS. 7 and 8). In order to initialise the augmented reality tracking, the subject is required to walk a few steps around the room while holding the device at an angle of approximately 60 degrees (see FIG. 9). After successful placement of all three objects (see FIGS. 10 and 11), the subject is instructed to pick the objects up again by pointing the device camera at the locations where they had placed the objects (see FIG. 12). Before the objects can be picked up, the subject is asked to walk back to the point where the object placement phase started (see FIG. 13).

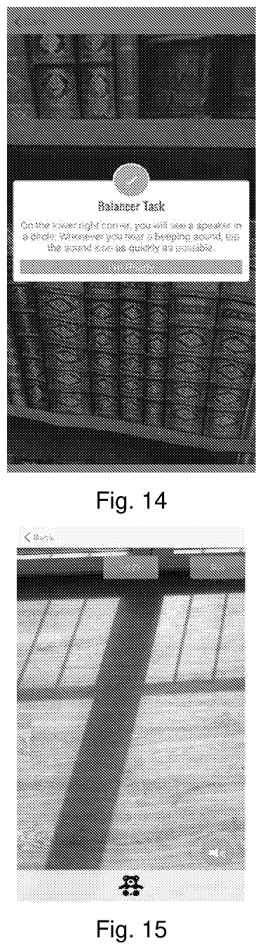

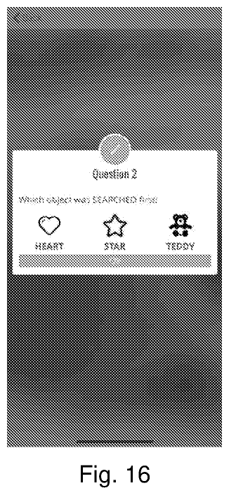

While picking up the objects, the subject has to react to audio signals (such as a high pitch beep sound), which prompt the subject to press a button on the bottom of the screen (see instructions in FIGS. 14 and 15). The sequence in which the subject is asked to pick up the objects is randomised in each run, while ensuring the last object placed is never the first object to be found. If the subject is not able to recall where one of the items was placed, they are able to skip the current object using the skip button (see FIG. 15). Once all objects have been found, skipped, or 3 minutes have passed, the test is concluded, and the subject has to answer two questions about the first objects that were placed and searched (see FIG. 16).

a. Day-Out-Task:

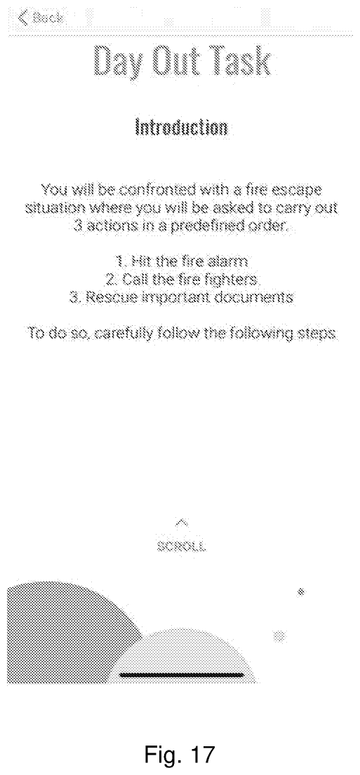

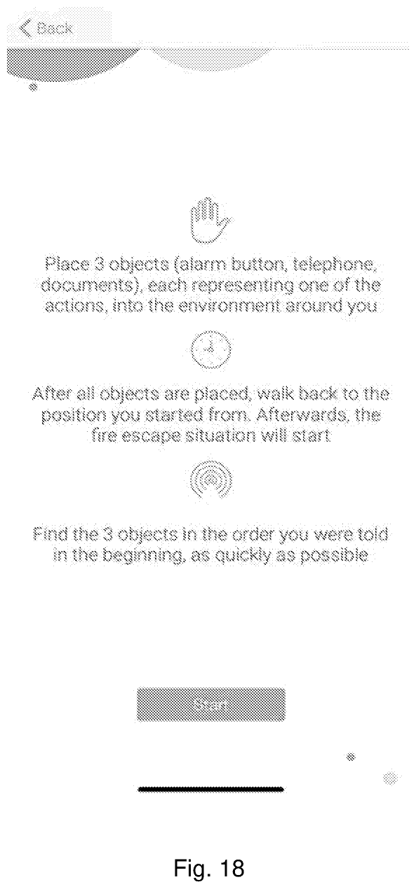

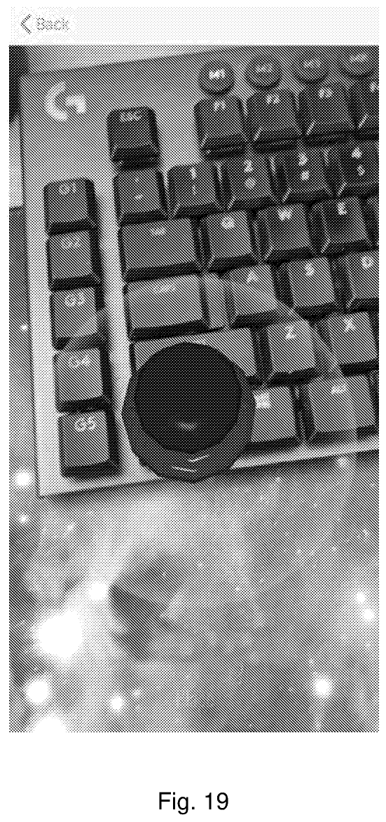

The Day-Out-Task uses a similar augmented reality functionality as the Back-in-Time task described above. The subject is confronted with a fire escape situation where three actions are to be carried out in a predefined order: 1) Trigger an alarm, 2) Call the firefighters; and 3) Rescue important documents (see FIGS. 17 and 18). To do so, the subject needs to place three objects representing the actions into their environment using augmented reality. Specifically, the subject needs to place 1) alarm button, 2) telephone, and 3) documents in their environment utilising the augmented reality functionality, similar to the Back-in-Time task above (see FIG. 19). The subject then carries out the tasks by picking up these three objects.

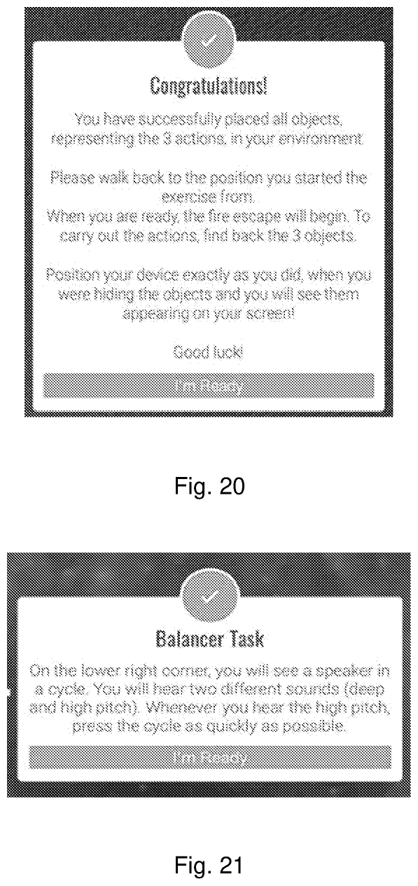

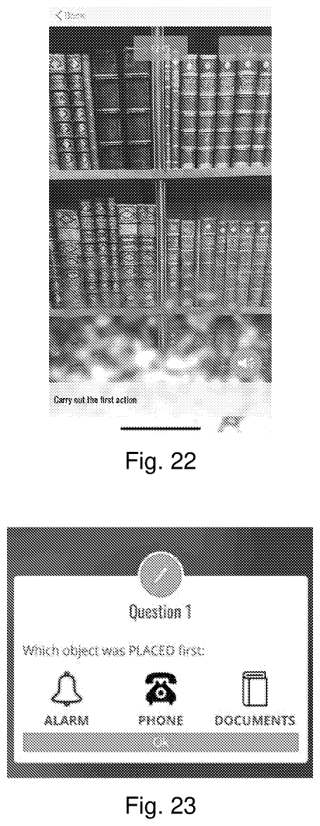

The sequence the subject is asked to place the objects in is randomised, while ensuring that the subject never places the alarm button last. The action sequence of picking up objects (carrying out tasks) is fixed as described above (see FIG. 20). Again, the subject is asked to react to the audio signal (such as beep sounds) while carrying out the action sequence (see FIG. 21). In contrast to the Back-in-Time task, the subject is asked to react only to audio signals (beep sounds) with a high pitch, while being presented with high and low pitch signals (see instructions in FIG. 21). While the objects need to be found, a fire animation engulfs the screen to mimic the urgency of a fire drill situation (see FIG. 22).

Once all tasks have been completed, skipped, or 3 minutes have passed, the test is concluded with two questions about the first object placed and the first object searched (see FIG. 23). The answer for the second question, which object was searched first, will (in contrast to the situation for the Back-in-Time task) always be the alarm button.

The system (preferably the software application) tracks response errors and reaction times of the subject. As described above, the device presents a combination of visual and auditory stimuli that is either sequential or simultaneous, depending on the task.

The subject is scored based on the timing and accuracy of their responses, as well as motion data, such as hand movement and walking patterns while the subject places and picks items in real space. This data is generated based on the device's sensor data.

### B. Principles of Operation

The above-described system enables automated characterisation of aspects of perceptual, neuro-motor, and memory function linked to human cortical information processing. The assessment is accomplished by tracking response errors and reaction times of the subject and recording the subject's ability to respond to variations of audio and visual stimuli. The test is rapid (taking only around 10 minutes), extensive (including many brain functional domains), and non-invasive (subject contact is limited to the portable electronic device).

The tasks described above define a set of activity measures (k1 to k8). The measures include:
(k1) spatial memory accuracy, measured as the number of correct items selected
(k2) planning accuracy, measured as the correct prospective memory task execution (exact order of subtasks)
(k3) omissions of the dual-task interactions between start and goal
(k4) perseverations of incorrect dual-task interactions while navigating to the goal
(k5) total time to complete the navigation route per item
(k6) upper extremity neuro-motor parameters, i.e. motion agility, speed, and smoothness of motion (derived from signal processing on the 3D Acceleration data provided by the iPad sensors) while completing the task
(k7) reaction time of ‘dual-task’ interactions, measured as the time elapsed between the stimuli and response
(k8) reaction time of ‘idle state’ measured as the time elapsed between the subject idle state and the next immediate interaction response to the ‘dual-task’ exercise (e.g. reaction time of the individual to the audible signals as appropriate)

In this Example, the measures k1 to k8 are used in a scoring algorithm to compute a functional impairment score. Specifically, from the data generated during the execution of the test, a total of 660 metrics are calculated, which belong to the set of activity parameters (k1-k8, above). The calculations involved are based on algorithms, including, but not limited to signal analysis, sensor-fusion, algebraic integration, Fourier analysis, and wavelet analysis. Given a database of metrics for multiple subjects, an algorithm can be trained to score new subjects based on these 660 metrics.

The resulting metrics are mapped to a total of nine cognitive domains (see Table 1).

TABLE 1
Cognitive domains analysed

| Cognitive Domain | Description |
|---|---|
| Perceptual Motor Coordination | Motor coordination in response to perceived input |
| Complex Attention | Capacity to choose what to pay attention to and |
| | what to ignore |
| Cognitive Processing Speed | Speed and accuracy of information processing |
| Inhibition | Ability to tune out stimuli that are irrelevant to the task |
| Flexibility | Ability to switch between thinking about two different |
| | concepts |
| Visual Perception | Visual search speed, visual perception and efficiency |
| Planning | Process of thinking about the activities required to |
| | achieve a desired goal |
| Prospective Memory | Ability to remember to carry out intended actions |
| | in the future |
| Spatial Memory | Ability to recognise items that previously appeared in |
| | physical space |

For each of the cognitive domains, a percentile rank score is calculated, which is adjusted for age and gender. The cognitive domain percentiles describe how many percent of the healthy population with the same gender, in the same age group performed worse than the current subject. Therefore, a value of 50% implies average performance and higher values imply above-average performance.

In addition, a single output measure or Score is provided. A Score of 0-50 implies that the subject belongs to the “impaired” class, while a Score above 50 implies the subject belongs to the “unimpaired” class. The information relating to the cognitive domain percentiles may be useful in some circumstances for interpreting the Score, for example, explaining why the Score might be very low in an individual case.

### C. Display of Results

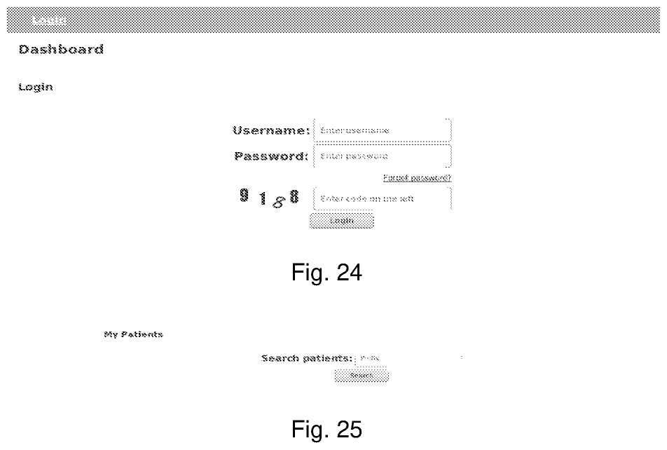

Test results for each subject can be accessed and reviewed by a medical practitioner using a dashboard (an example being the one provided by the applicant). An example is shown in FIG. 24.

When the practitioner enters the “My Patients” tab, a search field is presented as is shown in FIG. 25. After the user enters the Patient ID, the test results of the respective subject's sessions are presented.

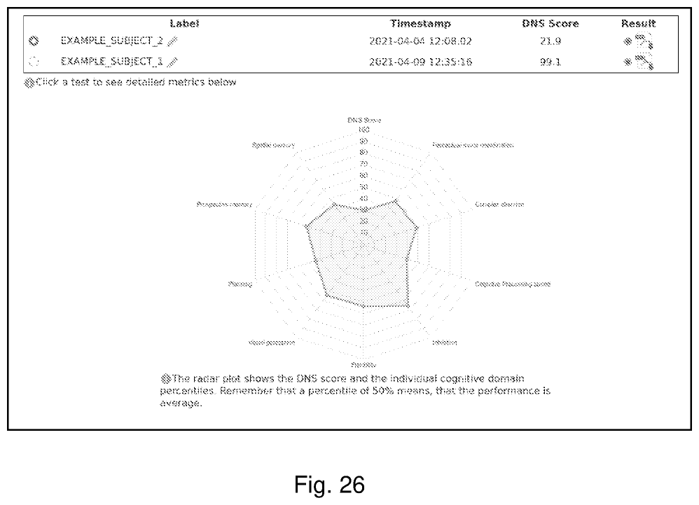

FIG. 26 provides an example of a subject result. On the right, in the table, a circle (red or green) is shown next to each entry. Where the Score is above 50, a green circle is shown (Subject 1). Where the Score is below 50, a red circle is shown (Subject 2). A Score above 50 indicates that the subject's performance is not correlated with the biological signature of Prodromal AD on the basis of @ amyloid aggregation (AB42/40 ratio), hence AD-related cognitive impairment is not probable. A Score below or equal to 50 indicates that the subject's performance is correlated with the biological signature of Prodromal AD on the basis of β amyloid aggregation (AB42/40 ratio), hence AD-related cognitive impairment is probable.

As shown in FIG. 26, once an entry in the table is selected (Subject 2 in this instance), further information is presented below the table as a radar plot, illustrating the Score on the top and each of the cognitive domain percentiles in a circular fashion around the plot.

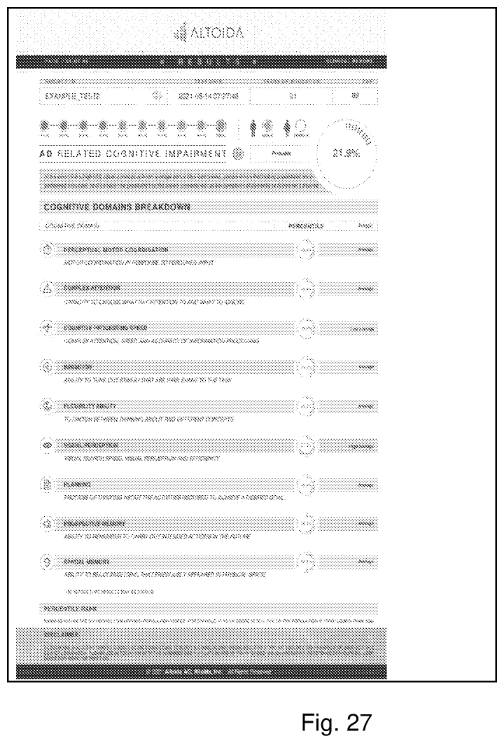

Next to the circle (red or green as appropriate), there is a PDF download icon that provides a downloadable PDF report. FIG. 27 shows an example of such a report (for Subject 2 from FIG. 26). Where the Score is below 50, the report will show “AD-related cognitive impairment: Probable”.

### D. Statistical Performance

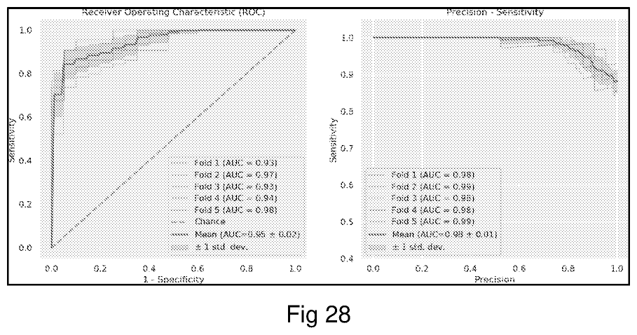

This classifier tests if it is possible to separate MCI subjects into either MCI/Ab− or MCI/Ab+, in other words, whether it is possible to detect the Amyloid beta status from MCI subjects. The performance of the classifier is plotted in FIG. 28. The model has been built using the following data of MCI patients:
Total dataset: 120 subjects, 426 datapoints
MCI/Ab−: 42 subjects, 101 datapoints
MCI/Ab+: 78 subjects, 325 datapoints

Statistics at Youden's optimal cutoff (0.81+−0.15) below (Table 2).

TABLE 2

| Statistic | Mean | S.D. |
|---|---|---|
| ROC - AUC | 0.95 | 0.02 |
| Accuracy | 0.86 | 0.05 |
| Precision/PPV | 0.99 | 0.01 |
| Sensitivity | 0.83 | 0.07 |
| Specificity | 0.96 | 0.02 |
| NPV | 0.65 | 0.08 |

### E. Validation

To evaluate technical success of the software, the machine learning algorithms were cross validated using the nested k-fold technique and demonstrated robust performance. Data collected to date also demonstrate an expectation of clinical success in providing a measurement of cognitive performance to aid in the assessment of impaired cognitive function for a physician to use in the diagnosis of AD. Specifically, Scores (impaired/unimpaired) have been correlated with readouts from the MMSE using bootstrapped Bayesian networks.

Connections between the digital measures obtained and cognitive features like MMSE were analysed via a recently developed Artificial Intelligence (AI) approach called Variational Autoencoder Modular Bayesian Networks (VAMBN) (Gootjes-Dreesbach et al. (2020), the contents of which are incorporated herein by reference). This is a hybrid of variational autoencoders and modular Bayesian Networks. In addition, the possibility of accurately predicting MMSE sub-item scores from the digital measures and vice versa via machine learning was tested.

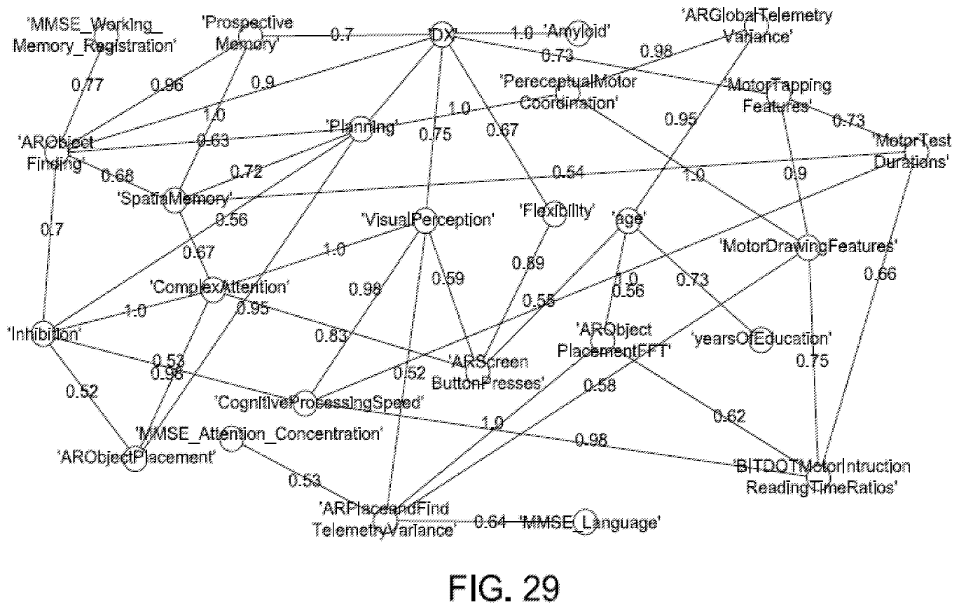

Digital measures within the AD Neuroimaging Initiative (ADNI) cohort were simulated and VAMBN was re-run. The application of VAMBN on the data from the virtual reality game resulted in a network comprising digital measures, MMSE sub-item scores and demographic features. FIG. 29 shows the variable dependencies identified in the dataset in 1000 bootstrapped Bayesian Network (BN) (strength>=0.5 and direction >=0.5) reconstructions. These edges indicate variable dependencies that are found commonly in bootstrapped BN reconstruction.

The network thus enabled disentanglement and quantification of the relationship between digital measures and established clinical scores. The simulation of digital measures and the application of VAMBN in the ADNI cohort enabled further prediction of connections of digital measures with features reflecting functional activities of daily living like FAQ (Functional Activity Questionnaire) and even molecular mechanisms. Two logistic regression binary classifiers were trained on data from virtual reality game and ADNI cohort in order to assess the sensitivity of digital measures to classify subjects into cognitively normal (CN) and mild cognitively impaired (MCI).

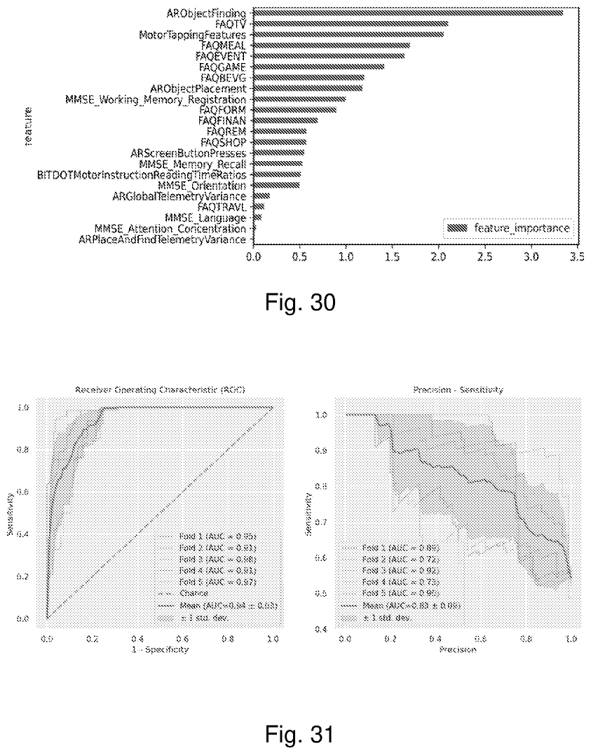

These results indicate that there is a significant dependency between digital measures and clinical scores such as MMSE and FAQ. Therefore, digital measures have the potential to act as a vital measure in the prediction of AD in a pre-symptomatic stage. Evaluation of the diagnostic benefit of digital measures led to the observation that they rank higher than some of the MMSE and FAQ features in their ability to classify patients into CN and MCI. FIG. 30 shows feature importance of MMSE, FAQ and DMs in ADNI data of the best logistic regression estimator to classify subjects into CN and MCI.

Additionally, studies have shown that the Score for MCI subjects detects the biological signature of Prodromal AD on the basis of β amyloid aggregation (AB42/40 ratio) with ROC-AUC>94%. (Bügler et al. (2020) and Tarnanas et al. (2015); the contents of which are incorporated herein by reference). This β amyloid aggregation signature has been suggested to predict MCI subjects who will convert to AD (Sörensen et al. (2020)).

### Example 2

In the above Example, eight activity parameters are measured to obtain the cognitive impairment score. In other embodiments, it is not necessary to measure all eight of the activity parameters. In an embodiment, a single parameter may be measured, and this may be upper extremity neuro-motor parameters (for example, motion agility, speed, and smoothness of motion while completing the task as defined in k6 of Example 1).

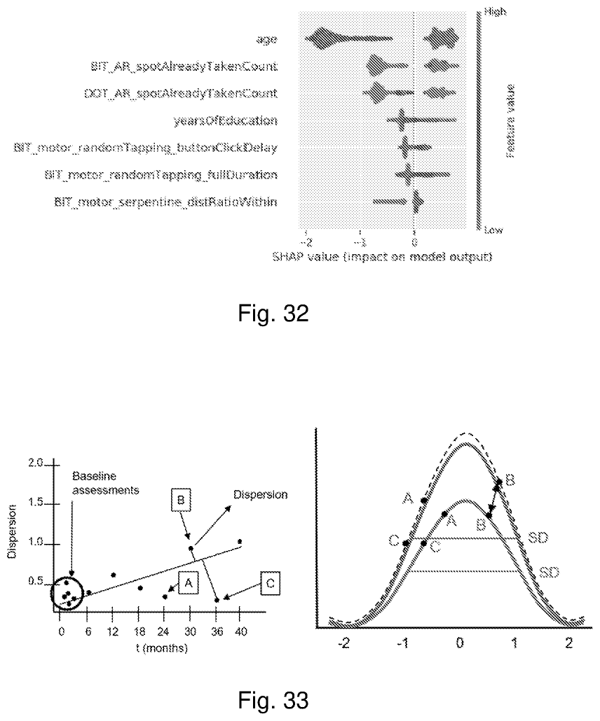

FIG. 31 shows ROC curves of classifier for this Example and FIG. 32 shows SHAP performance (impact on output). It can be seen that a useful functional impairment score can be obtained, even with a single activity parameter being measured.

Statistics at Youden's optimal cut-off (0.20+−0.12) below (Table 3).

TABLE 3

| Statistic | Mean | S.D. |
|---|---|---|
| ROC - AUC | 0.95 | 0.03 |
| Accuracy | 0.88 | 0.04 |
| Precision/PPV | 0.68 | 0.09 |
| Sensitivity | 0.97 | 0.01 |
| Specificity | 0.85 | 0.05 |
| NPV | 0.99 | 0.00 |

It was found that healthy controls could be distinguished from individuals with AD, prodromal AD and MCI/amyloid beta positive.

### Example 3

The above-described method and system (composite Score) was used in a study to measure individual-level change in AD. Dispersion measured with the Score system described above was compared to the conventional neuropsychological assessments for disease monitoring, characterising longitudinal risk trajectories, and predicting cognitive conversion events (from healthy to MCI and/or from MCI to AD).

### Methods

### Study Design

Two experiments (Study A and Study B) were conducted to assess the obtained Score against a set of established neuropsychological assessments as baseline. Study A (ClinicalTrials.gov Identifier: NCT02050464) was a semi-naturalistic observational study that included 29 participants, age 65+, with mild to moderate AD diagnosis recruited in Hirslanden Clinic, ZH, Switzerland. Study B (ClinicalTrials.gov Identifier: NCT02843529) was also a semi-naturalistic observational multicentre study which included 496 participants (213 MCI and 283 healthy controls (HC)), performed in ten European memory clinics and primary care centres, and two primary care community centres in the USA. Thus, a total of 525 participants enrolled in the two studies. These participants were either cognitively healthy (n=283), or diagnosed with MCI (n=213) or AD (n=29). The studies shared similar entry (inclusion/exclusion) criteria and clinical scales, and we characterised the AD biomarkers using the same criteria for the analysis. Both studies were approved by the local institutional review board (IRB), i.e., Bioethics committee of the Ionian University in Corfu, Greece where the studies were initiated.

In these studies, cognitive performance of the participants in three groups, namely HC, MCI, and AD, was measured using the composite Score as described above, and a set of traditional pencil-and-paper neuropsychological assessments. Thus, in this retrospective observational analysis, the independent variable is the testing method, composite Score as described above vs. neuropsychological assessments (elaborated under Materials), and our key dependent variable is dispersion.

### Participants

In both Study A and Study B, participants with any significant neurologic disease (such as Parkinson's disease, Huntington's disease, normal pressure hydrocephalus, brain tumour, progressive supranuclear palsy, seizure disorder, subdural hematoma, multiple sclerosis, or history of significant head trauma followed by persistent neurologic defaults or known structural brain abnormalities) were excluded at the recruitment stage. In Study B, further key inclusion criteria were: (1) 55-90 years of age, (2) fluency in English, French, Spanish, Greek, German or Italian, and (3) familiarity with digital devices, including currently possessing and actively using an iPad Pro or iPhone with an at-home Wi-Fi network for the remote assessments. Using these criteria, firstly a control group of 283 cognitively healthy individuals that underwent the same procedure at the Global Brain Health Institute (GBHI) at Trinity College, Dublin was recruited. In recruiting participants with cognitive impairments, the biomarkers (CSF, brain MRI and ApoE genotype) were used as a criterion, and cognitive deficits compatible with MCI diagnosis were found in 213 subjects: 170 from the memory clinics and primary care centres in various countries in Europe (detailed under Procedure section below) and 43 from the community centres in the USA. Seven participants were excluded from the data analysis due to poor data quality. The Study B cohort consisted of HC (n=283), and patients with MCI who are at high risk of developing AD within 18-40 months (n=213), assessed every 6 months. The MCI and AD cohorts were included independently on their biomarker status if their diagnosis was consistent with MCI and Alzheimer's dementia diagnosis according to core criteria of NIA-AA revised guidelines (Jack et al. (2011)). The participant cohort in Study B is further detailed in Bügler et al. (2020). The cohort in Study A (the symptomatic AD patients from the Hirslanden Clinic, Zurich, Switzerland) was added for control and comparison (n=29). Participants were matched on gender and educational level, with no statistically significant difference in cognitive performance between age groups on variables education (p=0.43, Cohen's d=0.4), or gender (p=0.68, Cohen's d=0.3).

### Procedure

Upon enrolment, all participants gave written informed consent for participation and for reuse of their data. In all groups (HC, MCI and AD), the composite Score test, as described above, was administered every 6 to 8 months over two days; Day 1 included training and a first measurement, and Day 2 included a ‘refresher training’ followed by a second measurement. One hundred participants used the composite Score method described above at home on Day 2 (these measurements were verified against those obtained in the clinic before inclusion in the analysis).

In overview, the procedure included:

1) A smart device incorporating the system disclosed herein was given to Primary Care Physicians and Memory Clinics for in-clinic assessments.

2) The tool was administered at baseline for two days: Day 1 (training and first measurement). Day 2 (refresh training and second measurement).

3) On some sites (Study B for healthy controls and participants with MCI), Day 2 was administered unsupervised at home in a total of n=100 subjects. These assessments showed the same performance as the Day 1 clinic visit.

4) The neuropsychological and test disclosed herein were administered at baseline and at follow-up every six months.

The first composite Score test duration was 20 minutes including training (10 minute training, 2 minute break, 8 minute measurement). After establishing this baseline, the composite Score test took an average of 8 minutes to administer every 6 to 8 months. The conventional neuropsychological assessment took between 120-140 minutes per visit, including breaks. Every 6 to 8 months, participants were also assessed for their clinical and neuropsychological status with the Mini-Mental State Examination (MMSE) or Montreal Cognitive Assessment (MOCA), and clinically examined if a transition from MCI to dementia (due to AD, or not associated with AD) occurred based on the diagnostic core criteria of NIA-AA (Jack et al. (2011)). Clinical outcomes for MCI/dementia/AD diagnoses were ascertained by investigators blinded to the predictor variables of this study.

Study A participants were tested for a total duration of 48 months between 2013 and 2017, and Study B participants for 40 to 42 months between 2017 and 2020. Participating memory clinics were in Greece, Italy, Spain, Ireland, Switzerland and the USA. Specifically, the following institutions enabled data collection for Study B: Greek Alzheimer's Association and Related Disorders “Ag. Giannis”, and “Ag. Eleni” memory clinics in Thessaloniki, Greece; the University of Roma La Sapienza memory clinic in Rome, Italy; IRCCS Centro San Giovanni di Dio Fatebenefratelli memory clinic in Brescia, Italy; Neuromed IRCCS memory clinic in Naples, Italy; Fundacion Clinic per a la Recerca Biomédica memory clinic in Barcelona, Spain; University of Dublin, Trinity College, St James memory clinic in Dublin, Ireland; BiHELab—Bioinformatics and Human Electrophysiology Lab and affiliated primary physicians' network in Corfu, Greece; two offices from the Practice for Personalized Medicine of the Hirslanden Private Hospital in Zurich & Aarau, Switzerland Scripps Health in La Jolla, California, USA; and the Center for Brain Health—The University of Texas at Dallas, USA.

### Materials

Baseline neuropsychological assessments. The baseline NP assessments included a comprehensive set of tests: the Wechsler Memory Scale (adjusted for education) (WMS-IV (2009)), MMSE (Folstein et al. (1975)) or MOCA (Nasreddine et al. (2005)), Clinical Dementia Rating (CDR) Memory Box score (Morris, 1993), and a full neuropsychological battery including the assessments Digit Span Forward, Digit Span Backward (WMS-IV (2009)), Trail Making Test A, Trail Making Test B (Butler et al., 1991), RAVLT Total, RAVLT A6, RAVLT A7 (RAVLT, 1996), Benton VRT (Benton Visual Retention Test Fifth Edition, 1991), Digit Symbol (Kaufman, 1983), Block Design (The Kohs Block-Design Tests.-PsycNET, 1932), Similarities (Drozdick et al., 2018), and Word and Animal Fluency (Benton, 1968). These tests, taken together, address 13 cognitive domains.

Overall Score. The digital biomarker data for cognition and functional abilities were collected using the composite Score as described above. The composite Score methodology selects the most promising indicators from previous work (such as those cited above) reducing the testing time from nearly two hours to ten minutes. It also contains new measures that have not been used in this context (e.g., measuring gait, touch pressure, walk path and tremor). This multivariate scoring increases the efficiency of digital phenotyping and enables better assessment of an individual's performance against their own history as well as against the ‘normative’ data based on other people in the same cohort. The composite Score as described above captures over 320 individual features, such as reaction time, speed, attention- and memory-based assessments, as well as every single device sensor input (or lack thereof) through accelerometer, gyroscope, magnetoscope, camera, microphone, and touch screen. The composite Score methodology as described above was tested in an independent pilot study with a sample of young, healthy controls across all the described cognitive domains, and found that test-retest variability was 0.156%. Such low variability shows excellent internal validity of the composite Score test and corroborates the representability and stability of its measures over time.

Additional biomarker tests. Additionally, AD biomarkers (β-amyloid and p-tau and total tau protein cerebrospinal fluid (CSF) levels, brain MRI and ApoE genotype) were collected as specific baseline measurements for the digital biomarkers obtained through composite Score test. To ensure a finer understanding of the type of cognitive impairment; classification in the diagnostic clusters of MCI and dementia due to AD (aMCI and ADD), or MCI and dementia not associated with AD (naMCI and nADD), were performed based on the β-amyloid and tau protein CSF levels biomarker.

### Statistical Analyses

To investigate variability in participants' cognitive performance, a common and meaningful index that can be compared between the composite Score as described above and gold standard neuropsychological assessments was obtained. For this, the so-called dispersion index was used (e.g., Hultsch et al., 2008; Wojtowicz et al., 2012). This was calculated for each individual based on their reaction times (including a control for speed-accuracy trade off) across cognitive measures within individuals and between healthy controls, MCI and AD groups (FIG. 33).

The dispersion index is a more reliable measure of central nervous system (CNS) integrity and of individual cognitive structure (Hultsch et al., 2008; Wojtowicz et al., 2012) than mean performance. Individual dispersion profiles are obtained by using a regression technique, which computes intra-individual standard deviation (iSD) scores from standardised test scores. Dispersion profiles were obtained for all cognitive domains measured by the composite Score test as described above and the neuropsychological test batteries used in the study to make them directly comparable. Test scores from the neuropsychological assessment battery were initially regressed on linear and quadratic age trends to control for group differences in mean performance. Controlling for group differences based on age is necessary because greater variance tends to be associated with greater means and mean-level performance which are expected to differ across age bands present in the study sample with participants in the age range of 55-90. The resulting residuals from these linear and quadratic regression models were standardised as T-scores (M=50, SD=10), and iSDs were subsequently computed across these residualised test scores. The resulting dispersion estimate, indexed on a common metric, reflects the amount of variability across an individual's neuropsychological profile relative to the group average (FIG. 33). The group average is obtained from participants' performance levels across measurements. Higher values in the dispersion index reflect greater intra-individual variability in cognitive function.

Next, longitudinal Risk Trajectory Scores (LTRS) and Longitudinal Decline Velocity Scores (LDVS) were computed (see below) across the 11 composite Score and the 13 conventional neuropsychological cognitive/functional domains.

For between-group mean comparisons, we used MANOVA and independent one-way ANOVA or T-test, whereas for within-group mean comparisons, we used independent one-way ANOVA. The Benjamini-Hochberg's correction for multiple testing was applied on all statistical analyses, using an alpha value of 0.05 (p<0.05, two-tailed). All statistical analyses were performed using SPSS 22.0 for Mac.

### Longitudinal Intra-Individual Variability-Related Metrics

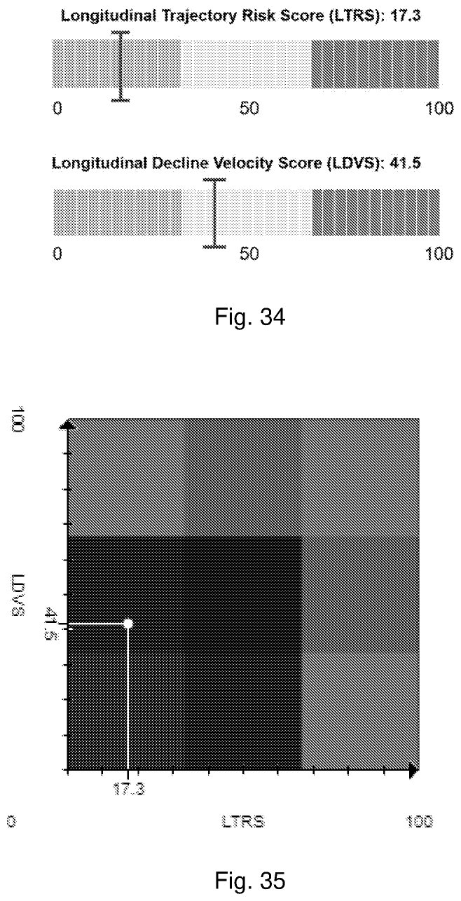

LTRS/LDVS The Longitudinal Trajectory Risk Score (LTRS) quantifies the changes on all cognitive domains, such as the amount of cognitive decline suffered by an individual, based on multiple linear regression models (FIG. 34, top). The LTRS does not take the period of time in which the decline occurs into account. It merely quantifies the magnitude of change that is captured by the observations. The Longitudinal Decline Velocity Score (LDVS), on the other hand, quantifies at what speed the change takes place, and thus can be used to assess whether decline is happening at a critical velocity in each of the cognitive domains (FIG. 34, bottom). The LDVS is also based on multiple linear regression models. A high value in the LDVS implies an unusually fast decline and builds a weighted linear regression model for each composite Score cognitive domain using simple linear regression with the rate of decline as “weight”. Preferably a participant performs at least four complete tests over a period of multiple weeks and the LTRS and LDVS can be interpreted together in a risk matrix (FIG. 35).

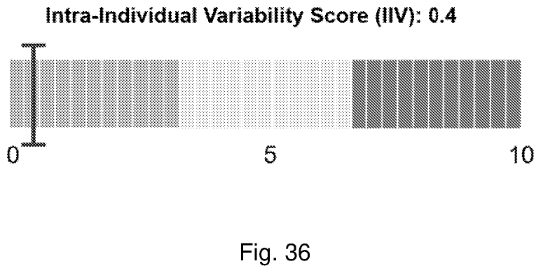

Intra-individual variability The intra-individual variability quantifies the fluctuation in cognitive performance of an individual and has been shown to sensitively detect underlying neural pathology of cognitive and functional change at the earliest stages of AD (FIG. 36).

The intra-individual variability quantifies the variability of cognitive domain percentiles over time. The value corresponds to the average variability of the subject's test in multiples of the variability of healthy subjects for each domain. Preferably at least five tests are done by the same participant. The intra-individual variability is a highly sensitive predictor of disease onset and conversion to AD.

### Results

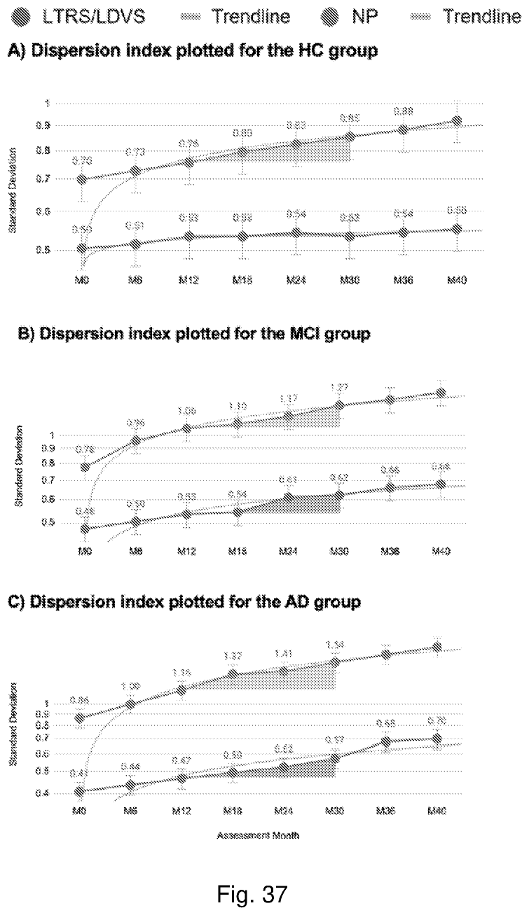

The dispersion score across the entire sample was 11.45 (SD=5.12) T-score units. FIG. 37 shows the magnitude of dispersion within each cognitive status subgroup (healthy controls, MCI, AD) based on LTRS and conventional neuropsychological assessments, demonstrating that the digital biomarkers explain up to 2.6 times more intra-individual variability compared to conventional paper-pencil neuropsychological assessments.

FIG. 37 shows dispersion index based on LTRS and neuropsychological tests plotted for the three different groups translated into Standard Deviation. The graphs show a non-linear increase in Standard Deviation as a function of disease trajectories. Comparing the overall mean of LTRS vs. NP per group yields the following values: HC: t=10.00106, p<0.00001; MCI: t=7.02195, p<0.00001; AD: t=6.65272. p=0.000011, the results are statistically significant at p<0.001. Details of the individual time points are shown in Table 4.

TABLE 4
LTRS/LVDS vs. neuropsychological trajectory differences and effect sizes (Cohen's d)

| HEALTHY | | | | | | MCI | | | | | | AD | | | | | |
|---|---|---|---|---|---|---|---|---|---|---|---|---|---|---|---|---|---|
| LTRS/LVDS | | p- | | 95% | | LTRS/LVDS | | p- | | 95% | | LTRS/LVDS | | p- | | 95% | |
| vs NP | [symbol] -value | value | Cohen's d | C.I. | | vs NP | [symbol] -value | value | Cohen's d | C.I. | | vs NP | [symbol] -value | value | Cohen's d | C.I. | |
| [symbol] | [symbol] | [symbol] | [symbol] | [symbol] | 1.4349 | [symbol] | [symbol] | [symbol] | 0.5742 | [symbol] | [symbol] | [symbol] | [symbol] | [symbol] | [symbol] | [symbol] | [symbol] |
| [symbol] | [symbol] | [symbol] | 1.4411 | [symbol] | [symbol] | [symbol] | [symbol] | 0.011 | 1.3675 | [symbol] | [symbol] | [symbol] | [symbol] | [symbol] | 1.771 | [symbol] | 2.288 |
| [symbol] | 12.50 | [symbol] | 1.4434 | [symbol] | [symbol] | [symbol] | [symbol] | 0.001 | 3.8071 | [symbol] | [symbol] | [symbol] | 7.25 | [symbol] | [symbol] | [symbol] | [symbol] |
| [symbol] | [symbol] | [symbol] | [symbol] | [symbol] | [symbol] | [symbol] | 11.52 | [symbol] | [symbol] | [symbol] | [symbol] | [symbol] | [symbol] | 0.001 | [symbol] | [symbol] | [symbol] |
| [symbol] | [symbol] | [symbol] | [symbol] | [symbol] | [symbol] | [symbol] | 11.23 | [symbol] | [symbol] | 1.2703 | [symbol] | [symbol] | [symbol] | [symbol] | [symbol] | [symbol] | [symbol] |
| [symbol] | 22.77 | [symbol] | [symbol] | [symbol] | [symbol] | [symbol] | [symbol] | [symbol] | [symbol] | [symbol] | [symbol] | [symbol] | 11.40 | [symbol] | [symbol] | [symbol] | [symbol] |
| [symbol] | 17.71 | [symbol] | [symbol] | [symbol] | [symbol] | [symbol] | [symbol] | [symbol] | [symbol] | 2.1013 | [symbol] | [symbol] | [symbol] | [symbol] | [symbol] | 2.755 | [symbol] |

[symbol]

indicates data missing or illegible when filed

Inferential analysis on the comparison of the values shown in FIG. 37 reveals that intra-individual variability differs between groups (F (2,522)=34.252, p<0.001, η²=0.25), where the AD group (m=23.78, SD=4.54) exhibits the highest variability, followed by the MCI (m=12.48, SD=2.91), and the HC (m=8.09, SD=1.64). Group differences across all domains were also observed based on the battery of 13 NP vs. 11 composite Score domains (Table 4). The AD group exhibited greater dispersion than MCI, and MCI greater than the HC (Table 4), verifying the robustness of measurements.

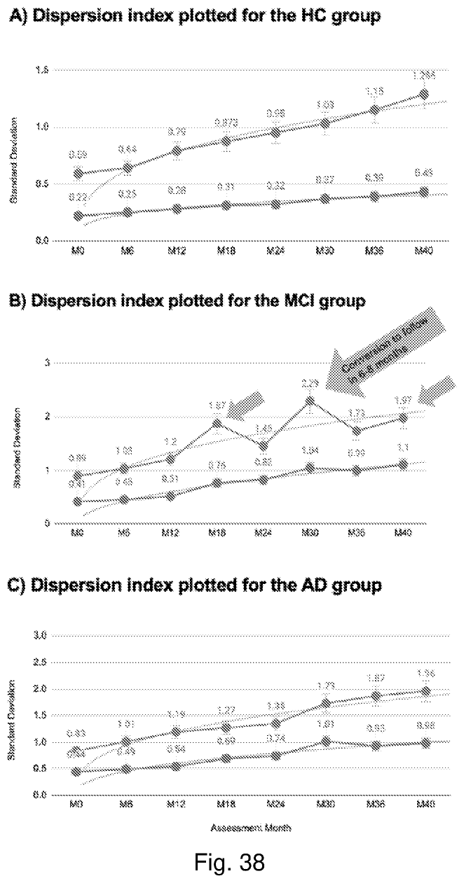

Following the LTRS/LDVS analysis, the longitudinal intra-individual variability for each group was also plotted revealing a non-linear increase in standard deviation as a function of disease trajectory (FIG. 38). In FIG. 38, intra-individual variability is consistently and significantly more sensitive at detecting disease trajectory trends than conventional neuropsychological assessments, especially with pre-conversion events (spikes in FIG. 38B predict a likely conversion from MCI to AD by next assessment). The ‘distance’ in dispersion measures increases between the two assessment types (longitudinal intra-individual variability and neuropsychological assessment) as the disease progresses, demonstrating that longitudinal intra-individual variability is more sensitive at detecting the markers than neuropsychological assessment (FIG. 38).

FIG. 38 shows dispersion index plotted across tasks, showing group intra-individual standard deviation (iSD) for the healthy controls (A), MCI (B) and AD (C) groups. The graphs show a non-linear increase in SD as a function of disease trajectories. Intra-individual variability is consistently and significantly more sensitive for the disease trajectory trends than conventional neuropsychological assessments, especially at the pre-conversion events (spikes in B predict a likely conversion by next assessment). LIIV: Longitudinal intra-individual variability.

Since the composite Score domain contains both LTRS and longitudinal intra-individual variability measures, the results shown in FIGS. 37 and 38 provide evidence of the strength of digital biomarkers. Longitudinal intra-individual variability is a strong preclinical risk predictor that determines conversion from MCI to AD (FIG. 38B) through machine learning algorithms, whereas the LTRS shows higher sensitivity at detecting change in cognition than conventional neuropsychological assessments (FIG. 37 and Table 4).

Taken together, these results demonstrate that the composite Score dispersion metric is consistently and significantly more sensitive at capturing disease trajectory trends than traditional neuropsychological assessments. In addition, the composite Score assessment allows for the prediction of conversion events 6 to 8 months prior to the conversion event. These conversion predictors are characterized by a spike in the intra-individual variability in the assessment prior to the actual conversion, illustrated in FIG. 38B, and are not detectable with conventional neuropsychological assessments.

### Discussion

In the above-described study, a persistent problem in cognitive aging research; the individual-level change in dementia with regards to cognition and function was tackled. Establishing when meaningful individual-level change has occurred is useful for evaluating dementia interventions, as well as for supporting lifelong brain health (Livingston et al. (2017)). The two metrics examined here in combination (LTRS/LDVS and longitudinal intra-individual variability integrated in the composite Score) may offer potential tools for practitioners. LTRS/LDVS at the individual level may be useful to assess the efficiency of cognitive training, medication or remediation, and it is a valuable alternative to the more frequently used Reliable Change Index. Provided the frequency of data collection is sufficient, LTRS/LDVS makes it possible to assess individual changes in performance more sensitively than conventional paper-pencil assessments, and without the inconvenience of having to compare with change in a normative sample subject to inter-individual variability issues. Also, unlike the traditional Reliable Change Index, longitudinal intra-individual variability offers a reliable tool to draw conclusions solely based on individual performance. This may be particularly valuable in the context of adaptive trials that utilise information on an ongoing basis for the purposes of maximising trial efficiency, as well as for early detection of disease progression events, including those in the prodromal phase of dementia (Ritchie et al. (2016)).

In the context of AD, dispersion has been shown to be a sensitive marker to detect change in cognition and functional abilities even at prodromal stages of the disease (Hultsch et al., 2008; Wojtowicz et al., 2012). Establishing meaningful change at the level of an individual is instrumental, as significant effects in group-level statistics do not show (and cannot even imply) what changes have occurred for any one individual (Murray et al. (2021)). Taking both of these facts into account, dispersion differences between a full 120-140 min conventional NP assessment with 13 cognitive domains and the 10 minute composite Score assessment with 11 cognitive domains, were analysed and the dispersion in a group of HC, MCI and AD participants over 40 months was compared. The composite Score showed consistently and significantly higher sensitivity in capturing these changes for disease trajectory trends. This was particularly true at later stages of the disease, as shown in LTRS/LDVS results (FIG. 2, Table 1), likely due to the complex domains integrating function and cognition uniquely in the composite Score.

These findings demonstrate that the composite Score methodology as described above is a useful tool for disease progression monitoring as well as for clinical trial endpoints. Further, intra-individual variability was consistently more sensitive at identifying markers of disease trajectory trends than the conventional NP assessment (FIG. 38). Intra-individual variability was also a particularly strong marker among the composite Score metrics at detecting pre-conversion events, rendering the tool capable of predicting conversion from MCI to AD 6-8 months prior to the actual event. Such a prediction not only allows for lifestyle interventions to delay conversion and maintain a healthier brain for longer, but also gives patients and family time for preparation, care adjustment, and pharmacological intervention once available. Additionally, this approach can allow for prospective and longitudinal assessments of biological (imaging, genetic, biochemical) and functional markers implicated in the pathophysiology of dementia. This should help lead to a greater understanding of development and onset of the disease. It has not previously been possible to predict transition events from MCI to symptomatic AD months in advance.

The composite Score methodology differs from the conventional neuropsychological assessments in that it captures multidimensional digital biomarkers and it is not limited to latency- or accuracy-based measures. It integrates several objectively measured features into a single task. This integration increases the ecological validity of the observations, as it creates a more generalisable ‘real-world situation’ than the traditional laboratory test-settings. It is unsurprising that the abundance of data collected by composite Score method both by the novel combination of multiple variables addressing, in an embodiment, 11 cognitive domains as well as sensor data yields a higher sensitivity, particularly when variability measures are considered. The composite Score digital biomarker platform produces significant volumes of high-resolution data that include cognitive and motor processing; voice-based data that are indicative of the affective state and micro-errors that divulge where, when, and how a disease manifestation is affecting everyday function. These data have the potential to be further leveraged for disease progression modelling, for more accurate conversion event prediction or modelling of drug effects, leading to at-scale, non-intrusive lifelong monitoring of brain health.

It is important to note that both dispersion and intra-individual variability exhibit a non-linear increase with age. Current patterns of data reveal that greater dispersion across domains is associated with poorer cognitive performance, possibly reflecting reduction in cognitive control. The spikes of intra-individual variability in the MCI group are potentially explained by the demands of executive function, a domain particularly affected in MCI, due to the complexity of the composite Score assessment, in addition to internal and external factors such as anxiety and depression that particularly affect this disease stage.

Another important feature of the composite Score method described above is its efficiency. It takes 10 minutes to administer the composite Score test as opposed to a 120 minute conventional neuropsychological test battery, and it yields highly comparable results, even when administered at home (as opposed to during a clinic visit). Also, heterogeneity/homogeneity features of the composite Score and LTRS/LDVS or longitudinal intra-individual variability changes in diverse cognitive abilities may also be a valuable tool for clinicians.

The findings in this study highlight the sensitivity of digital biomarkers at detecting changes in cognition, and open interesting directions for research concerning heterogeneity in cognitive change.

This study demonstrates that active digital biomarkers are useful tools for monitoring disease progression in cognitive aging. Such tools could be used by primary caregivers without much training in dementia testing to refer patients for further testing, or to provide necessary resources to mitigate debilitating effects of cognitive decline. This study's findings are also relevant to clinical trials, as the prediction of AD conversion 6 to 8 months prior to the event may allow the detection of meaningful change that could also influence the dosage of medication, and permit closer patient monitoring. In addition, observing such changes early enables the study of underlying disease markers immediately prior to conversion, contributing to increased understanding of pathophysiological processes of AD and the possible discovery of new phenotypes of cognitive decline.

### CONCLUSION

This work represents the first attempt to explore active digital biomarkers, such as those included in the composite Score method described above, for detecting meaningful change based on newly utilised metrics at the individual level. While mean scores of cognitive tests are important for disease characterisation, the intra-individual variability across tests harbours large amounts of information that can easily be captured. Novel metrics using smart-device sensors show an increased sensitivity compared to conventional neuropsychological assessments. The composite Score method described above has been found to be 2.6× more sensitive than a conventional battery for dementia and takes only ten minutes. This “better” and “faster” performance renders the composite Score method an exceptional tool for patient care and can also be used to determine when an individual has undergone meaningful change in symptoms for monitoring drug interventions.

### Example 4

An individual previously diagnosed with MCI carries out the above-described tests using the composite Score system on their smartphone at home on a monthly basis, and even more frequently if desired or appropriate. Their performance results in a composite Score assessment made up of a Longitudinal Trajectory Risk Score (LTRS) and a Longitudinal Development Velocity Score (LDVS). The composite Score measures therapeutic response and how this translates into cognitive and functional improvements in everyday function. It can be computed monthly right after a therapeutic intervention with an agent, such as Aduhelm and adds together the score from LTRS and LDVS. The range is 0-200 and a proposed visualisation for the composite Score is shown at FIG. 35.

In this implementation, LTRS takes longitudinal progress of each cognitive domain over a given time window of six months. It then builds a linear regression model for each cognitive domain using simple linear regression. The result is a line equation y=ax+b for each cognitive domain i: y_i=m_i*t+b_i. Initialize LTRS=0 for each cognitive domain i. If the slope of the linear model is negative (i.e. there is decline), add the absolute value of the slope to LTRS. If LTRS>100, set LTRS=100. LTRS scores range from 0-100. When the window from the therapeutic intervention is less than 6 months, the LTRS scores are used for calibration. Similarly, LDVS in this implementation takes longitudinal progress of each cognitive domain over a given time window of one month. It then builds a linear regression model for each cognitive domain using simple linear regression. The result is a line equation y=ax+b for each cognitive domain i: y_i=m_i*t+b_i. When the slope of the linear model is negative at critical velocity (i.e. there is decline within 1 month), the intra-individual standard deviation (iSD) is calculated as the standard deviation across standardised scores of the calculated cognitive domains for a single individual, as multiplier for the absolute value of the LDVS slope. Both LTRS and LDVS values are added together to create the composite Score (0-200).

In this Example, a composite Score of 150-200 means that the therapeutic intervention is working, and no adjustment needs to be made.

A composite Score between 100 and 150 alerts the physician to look further into the calculated cognitive domain percentiles for the different cognitive domains (for example the nine cognitive domains listed in Table 1) and the system suggests to the individual's physician an appropriate intervention to improve/prevent further deterioration in that cognitive domain. For example, if the patient shows a low score in visuospatial function, then the system may suggest prescription of memantine (donazepil).

When the composite Score is 50-100 the system may suggest an increase in the therapeutic agent. In an example, the therapeutic agent may be Aduhelm, which is an amyloid beta-directed antibody indicated for the treatment of AD. In another example it could be AADvac1, which is a compound effective against harmful tau protein aggregations in the brain and is linked to slower accumulation of a neurofilament light-chain (NfL) protein in one placebo-controlled randomised phase 2 study, suggesting slower neurodegeneration compared to the patients who received the placebo (Novak et al. (2021)).

Further to this, the physician is also encouraged to look further into the calculated cognitive domain percentiles for the different cognitive domains (for example the nine cognitive domains listed in Table 1) and the system suggests to the individual's physician an appropriate intervention to improve/prevent further deterioration in that cognitive domain. For example, Metformin for executive function, a metabolism/bioenergetic compound currently at Phase 3 or TRx0237 for perpetual motor coordination, a Tau-directed antibody compound currently at Phase 3 (Cummings et al. (2020).

Finally, when the composite Score is less than 50 then the system might suggest that the therapeutic intervention is at a critical stage or failing for this particular individual/patient.

The system can thus be used to diagnose an individual with mild cognitive impairment or AD or to predict whether an individual with mild cognitive impairment will convert to AD in due course. It can also be used to assist a physician with prescribing appropriate interventions and/or help to determine whether an already prescribed intervention is working. The system may therefore assist a physician by suggesting starting an intervention, stopping an intervention, or changing an intervention, pharmaceutical or otherwise. It may suggest an appropriate frequency and/or dose of a pharmaceutical intervention or specific drug to be administered to the individual and/or may suggest an appropriate route of administration of a pharmaceutical intervention for that individual. This applies to the specific pharmaceutical interventions mentioned above, for example in Example 4, and to all other potential pharmaceuticals whether or not disclosed herein.

One of the significant advantages of the system described herein is that it is able to assess cognitive capabilities in a single test as compared to the standard neurophysiological assessments currently used in diagnosing AD. As a result, cognitive function measurements can be administered in approximately 10 minutes as compared to 2 hours for the traditional neurophysiological assessments (e.g., MMSE, ADAS-Cog).

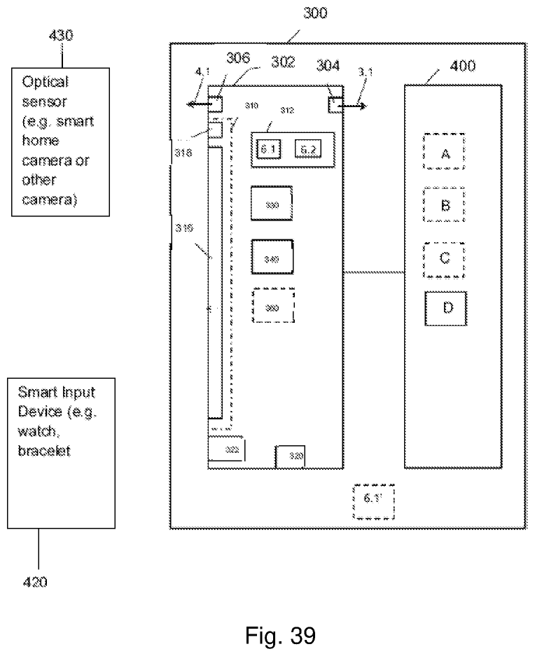

An example of apparatus 300 for use in implementing the teachings herein is shown in FIG. 39. The apparatus 300 in this example comprises a mobile device 302, provided with first and second cameras 304, 306, typically one being front facing (away from the user) and the other being rear facing (towards the user and the same side as the display). The mobile device 300 typically also includes an output unit 310, a position sensor 312 (such as a GPS module, an accelerometer and so on), a microphone 320, a user input unit 322 and one or more processing units 330, 340, 360.

The mobile device 300 is preferably a handheld portable device like a smartphone. However, the mobile device 302 may also be any other user portable device. It may, for example, be wearable, such as a smart watch or bracelet, smart glasses or similar. The mobile device 300 may be a single device or implemented in a plurality of devices, such as a smart telephone in conjunction with a smart watch or bracelet, or even glasses. FIG. 39 shows such smart devices 420 as external accessories configured to communicate with the mobile device 300.

The output unit 310 may include a display 316 and in some implementations a projector, such as an eye projector in a pair of smart glasses. The output may also include an acoustic unit 318 such as a loudspeaker and/or audio output port for earphone or headphones.

There may be provided an internal device 400, typically a processing unit, advantageously an artificial neural network, for carrying out computational work remote from the mobile device 300, including but not limited to computation of data from a plurality of different subjects, as provided for in the above teachings. The processing unit 400 would typically be coupled to the mobile device 300 to exchange data, remotely such as through the internet, a wireless network or via the GSM network. In some implementations the processing until 400 may comprise a central processing computer. It is to be appreciated that in some embodiments all processing is carried out within the device 300.

The apparatus may also include, as described above, an external optical sensor such as a smart home camera or other camera 430 configured to obtain images of the subject and relaying them either to the mobile unit 300 or to the external processing unit 400 or to both. It will be appreciated that the external optical unit 430 may comprise a set of cameras or the like, able to obtain a plurality of images of a subject, whether sequentially or simultaneously.

The skilled person will appreciate that this is just one example of device to implement the teachings herein and will understand from these teachings how to configure or construct a different electronic device to perform the same tasks.

The above-described method and system provide a measurement of cognitive performance to aid in the assessment of impaired cognitive function for a physician to use in the diagnosis of AD. They are intended for use preferably as an assessment aid and are not intended to identify the presence or absence of an AD diagnosis. In particular, they may be used as an adjunct to other diagnostic evaluations, and are intended to predict conversion from MCI to AD in subjects previously diagnosed with MCI. However, they may find use in assessing cognitive function and/or prediction of developing dementia attributable to other conditions.

It will be appreciated by those skilled in the art that changes could be made to the embodiments and examples described above without departing from the broad inventive concept. It is understood, therefore, that this invention is not limited to the particular embodiments disclosed, but it is intended to cover modifications within the spirit and scope of the present invention as defined by the claims.

All features disclosed and described with respect to the method may be used with the system, and vice versa.

The disclosures in United States patent application number U.S. 63/211,953, from which this application claims priority, and in the abstract accompanying this application are incorporated herein by reference.

### REFERENCES

Bäckman et al. (2001) Brain 124, 96-102
Benton (1968) Neuropsychologia 6, 53-60
Benton VRT (1991) Benton Visual Retention Test Fifth Edition
Blennow et al. (2006) Lancet 368, 387-403
Bügler et al. (2020) Alzheimer's Dement. 12, e12073
Butler et al. (1991) Professional Psychology: Research and Practice, 22, 510-512
Cummings et al. (2020) Alzheimer's Dement. 6, e12050
DeKosky & Marek (2003) Science 302, 830-4
Drozdick et al. (2018) The Wechsler Adult Intelligence Scale-Fourth Edition and the Wechsler Memory Scale-Fourth Edition. In Contemporary intellectual assessment:
Theories, tests, and issues, 4th ed (pp. 486-511). The Guilford Press.
Dubois et al. (2007) Lancet Neurol. 6, 734-46
Folstein et al. (1975) Journal of Psychiatric Research 12, 189-98
Forsberg et al. (2008) Neurobiol. Aging 29, 1456-65
Gootjes-Dreesbach et al. (2020) Front. Big Data 3, 16
Hultsch et al. (2008) The Handbook of Aging and Cognition 3rd Ed.
Jack et al. (2011) Alzheimer's & Dementia 7, 257-62
Kaufman (1983) Journal of Psychoeducational Assessment 1, 309-13
Kourtis et al. (2019) NPJ Digit. Med. 2, 9
Livingston et al. (2017) Lancet 390,2673-734
Morris (1993) Neurology 43, 2412
Mungas et al. (2010) Psychology and Aging 25, 606-619
Murray (2021) Alz. Res. Therapy 13, 26
Nasreddine et al. (2005) Journal of the American Geriatrics Society 53, 695-9
Novak et al. (2021) Nat. Aging 1, 521-34
Nordberg (2007) Curr. Opin. Neurol. 20, 398-402
Palmer et al. (2002) Am. J. Psychiatry 159, 436-42
RAVLT: Rey Auditory Verbal Learning Test (1996) WPS
Ridha et al. (2007) Arch. Neurol. 64, 849-54
Ritchie et al. (2016) Lancet Psychiatry 3, 179-86.
Sörensen et al. (2020) Alzheimers Res. Ther. 12, 155.
Tarnanas et al. (2015) Alzheimer's & Dementia: Diagnosis, Assessment & Disease Monitoring 1, 521-32
The Kohs block-design tests (1932) A revision for clinical practice.-PsycNET WMS-IV Wechsler Memory Scale 4th Edition. (2009)
Wojtowicz et al. (2012) International Journal of MS Care 14, 77-83
WO 2010/075481
WO 2016/157093
WO 2020/049470

1. A method of obtaining a measurement of cognitive performance in an individual, the method including:
obtaining a measure of the following activity parameters for the individual:
1) spatial memory accuracy;
2) ability to carry out dual-task interactions while navigating to a goal, wherein omission of the dual-task interactions is measured;
3) perseverations of incorrect dual-task interactions while navigating to a goal;
4) upper extremity neuro-motor parameters derived from signal processing of 3D acceleration data provided on a portable electronic device;
5) reaction time of dual-task interactions; and one or more of:
6) planning accuracy;
7) total time required by the individual to complete a navigation route; and
8) reaction time of the idle state of the individual;
receiving the measures obtained into an algorithm;
calculating a plurality of metrics belonging to the activity parameters; the metrics being mapped to a plurality of cognitive domains; and calculating a percentile rank score for each cognitive domain on the basis of baseline measurements obtained from a population of healthy individuals; and
computing a functional impairment score for the individual on the basis of the individual's percentile rank scores for the cognitive domains.

2. A method as claimed in claim 1, wherein the measurements are obtained using an app on an electronic portable device.

3. A computer implemented system for obtaining a measurement of cognitive performance in an individual, said system including:
an input interface configured to receive measurements from a remote source on an individual in respect of at least one of the following activity parameters:
1) spatial memory accuracy;
2) ability to carry out dual-task interactions while navigating to a goal, wherein omission of the dual-task interactions is measured;
3) perseverations of incorrect dual-task interactions while navigating to a goal;
4) upper extremity neuro-motor parameters derived from signal processing of 3D acceleration data provided on a portable electronic device;
5) reaction time of dual-task interactions; and one or more of:
6) planning accuracy;
7) total time required by the individual to complete a navigation route; and
8) reaction time of the idle state of the individual;
receiving the measures obtained into an algorithm;
a processor configured to receive said measurements and configured to execute computer program code to compute, using said measurements, a plurality of metrics belonging to the activity parameters; the metrics being mapped to a plurality of cognitive domains; and to calculate a percentile rank score for each cognitive domain on the basis of baseline measurements obtained from a population of healthy individuals; and to compute therefrom a functional impairment score indicative of cognitive performance in the individual based on the individual's percentile rank scores for the cognitive domains.

4. A computer implemented system as claimed in claim 3, said system including:
an input interface configured to receive a first measurement or set of measurements obtained at a first time and a second measurement or set of measurements obtained at a second different time in respect of the activity parameter or parameters;
a processor configured to receive said first measurement or set of measurements and configured to execute computer program code to compute, using said first measurement or set of measurements, a first functional impairment score indicative of cognitive performance in the individual, and configured to receive said second measurement or set of measurements and configured to execute computer program code to compute, using said second measurement or set of measurements, a second functional impairment score;
the processor configured to compare said second functional impairment score with said first functional impairment score and determine a magnitude and/or a speed of change in said functional impairment scores.

5.-6. (canceled)

7. A system as claimed in claim 4, wherein the processor or the system is configured to determine both a magnitude and a speed of change in the functional impairment scores for that individual to calculate a composite score.

8. A method as claimed in claim 2, wherein a system configured to receive said measurements and compute the functional impairment score and/or the composite score is remote from the electronic portable device or user device.

9. A method as claimed in claim 2, wherein a plurality of activity parameters is measured.

10. A method as claimed in claim 2, wherein at least the upper extremity neuro-motor parameters are measured.

11.-13. (canceled)

14. A method as claimed in claim 2, wherein all eight of the activity parameters are measured.

15. (canceled)

16. A method or system as claimed in claim 1, wherein the metrics are calculated on the basis of an algorithm.

17. A method as claimed in claim 16, wherein the algorithm is or includes one or more of signal analysis, sensor-fusion, algebraic integration, Fourier analysis or wavelet analysis.

18. A method as claimed in claim 17, wherein the cognitive domains include at least one of perceptual motor coordination, complex attention, cognitive processing speed, inhibition, flexibility, visual perception, planning, prospective memory, and spatial memory.

19. A method or system as claimed in claim 2, wherein hand movements of the individual are assessed to obtain at least one of the measurements.

20. A method as claimed in claim 19, wherein assessing the individual's hand movements includes testing speed and/or accuracy of the individual's hand movements.

21. A method as claimed in claim 19, wherein the individual's hand movements are assessed by displaying an image to the individual and assessing the individual's ability to trace or tap on the image.

22. A method as claimed in claim 21, wherein the image is displayed on the screen of a portable electronic device or other user device.

23. A method or system as claimed in claim 2, wherein the individual's ability to navigate is assessed to obtain at least one of the measurements.

24. A method as claimed in claim 23, wherein assessing the individual's ability to navigate includes the individual placing and retrieving a plurality of objects.

25. A method as claimed in claim 2, wherein the individual's ability to execute tasks is assessed to obtain at least one of the measurements.

26. A method as claimed in claim 2, wherein assessing the individual's ability to execute tasks includes assessing their ability to carry out subtasks in an exact order.

27.-52. (canceled)
